SEQUENCE LISTING (39 sequences)

SEQ ID NO: 1 - protein, 5 aa, Artificial Sequence
  Synthetic polypeptide
  MISC_FEATURE (1)..(1): Xaa is T or D
  MISC_FEATURE (2)..(2): Xaa is Y or F
  MISC_FEATURE (3)..(3): Xaa is N or Y
  MISC_FEATURE (4)..(4): Xaa is V, I, or M
  MISC_FEATURE (5)..(5): Xaa is H or N
XXXXX

SEQ ID NO: 2 - protein, 5 aa, Artificial Sequence
  Synthetic polypeptide
  MISC_FEATURE (4)..(4): Xaa is V or I
TYNXH

SEQ ID NO: 3 - protein, 5 aa, Artificial Sequence
  Synthetic polypeptide
TYNVH

SEQ ID NO: 4 - protein, 5 aa, Artificial Sequence
  Synthetic polypeptide
TYNIH

SEQ ID NO: 5 - protein, 5 aa, Artificial Sequence
  Synthetic polypeptide
DFYMN

SEQ ID NO: 6 - protein, 19 aa, Artificial Sequence
  Synthetic polypeptide
  MISC_FEATURE (1)..(1): Xaa is F or absent
  MISC_FEATURE (2)..(2): Xaa is I or absent
  MISC_FEATURE (3)..(3): Xaa is R or absent
  MISC_FEATURE (4)..(4): Xaa is A or N
  MISC_FEATURE (5)..(5): Xaa is M or K
  MISC_FEATURE (6)..(6): Xaa is W or V
  MISC_FEATURE (9)..(9): Xaa is G or Y
  MISC_FEATURE (10)..(10): Xaa is R or G or T
  MISC_FEATURE (15)..(15): Xaa is S or P
  MISC_FEATURE (16)..(16): Xaa is A or S
  MISC_FEATURE (17)..(17): Xaa is F or V
  MISC_FEATURE (19)..(19): Xaa is S or G
XXXXXXNGXXTDYNXXXKX

SEQ ID NO: 7 - protein, 16 aa, Artificial Sequence
  Synthetic polypeptide
  MISC_FEATURE (7)..(7): Xaa is R or G
AMWNGGXTDYNSAFKS

SEQ ID NO: 8 - protein, 16 aa, Artificial Sequence
  Synthetic polypeptide
AMWNGGRTDYNSAFKS

SEQ ID NO: 9 - protein, 16 aa, Artificial Sequence
  Synthetic polypeptide
AMWNGGGTDYNSAFKS

SEQ ID NO: 10 - protein, 19 aa, Artificial Sequence
  Synthetic polypeptide
FIRNKVNGYTTDYNPSVKG

SEQ ID NO: 11 - protein, 11 aa, Artificial Sequence
  Synthetic polypeptide
  MISC_FEATURE (1)..(1): Xaa is E or absent
  MISC_FEATURE (2)..(2): Xaa is D or absent
  MISC_FEATURE (3)..(3): Xaa is Y or absent
  MISC_FEATURE (4)..(4): Xaa is Y or absent
  MISC_FEATURE (5)..(5): Xaa is Y or absent
  MISC_FEATURE (6)..(6): Xaa is S or absent
  MISC_FEATURE (7)..(7): Xaa is G or absent
  MISC_FEATURE (8)..(8): Xaa is R or absent
  MISC_FEATURE (9)..(9): Xaa is L or P or F
  MISC_FEATURE (10)..(10): Xaa is D or T
  MISC_FEATURE (11)..(11): Xaa is D or Y
XXXXXXXXXXX

SEQ ID NO: 12 - protein, 11 aa, Artificial Sequence
  Synthetic polypeptide
EDYYYSGRFDY

SEQ ID NO: 13 - protein, 11 aa, Artificial Sequence
  Synthetic polypeptide
  MISC_FEATURE (5)..(5): Xaa is S or N
  MISC_FEATURE (6)..(6): Xaa is V or I
  MISC_FEATURE (7)..(7): Xaa is G or N
  MISC_FEATURE (8)..(8): Xaa is I or K
  MISC_FEATURE (9)..(9): Xaa is D or N or Y
  MISC_FEATURE (10)..(10): Xaa is V or L
KASQXXXXXXD

SEQ ID NO: 14 - protein, 11 aa, Artificial Sequence
  Synthetic polypeptide
  MISC_FEATURE (9)..(9): Xaa is D or N
KASQSVGIXVD

SEQ ID NO: 15 - protein, 11 aa, Artificial Sequence
  Synthetic polypeptide
KASQSVGIDVD

SEQ ID NO: 16 - protein, 11 aa, Artificial Sequence
  Synthetic polypeptide
KASQSVGINVD

SEQ ID NO: 17 - protein, 11 aa, Artificial Sequence
  Synthetic polypeptide
KASQNINKYLD

SEQ ID NO: 18 - protein, 7 aa, Artificial Sequence
  Synthetic polypeptide
  MISC_FEATURE (1)..(1): Xaa is G or H
  MISC_FEATURE (2)..(2): Xaa is A or T
  MISC_FEATURE (3)..(3): Xaa is S or N
  MISC_FEATURE (4)..(4): Xaa is S or N
  MISC_FEATURE (5)..(5): Xaa is R or L
  MISC_FEATURE (6)..(6): Xaa is H or T
XXXXXXT

SEQ ID NO: 19 - protein, 7 aa, Artificial Sequence
  Synthetic polypeptide
  MISC_FEATURE (4)..(4): Xaa is N or S
GASXRHT

SEQ ID NO: 20 - protein, 7 aa, Artificial Sequence
  Synthetic polypeptide
GASNRHT

SEQ ID NO: 21 - protein, 7 aa, Artificial Sequence
  Synthetic polypeptide
GASSRHT

SEQ ID NO: 22 - protein, 7 aa, Artificial Sequence
  Synthetic polypeptide
HTNNLQT

SEQ ID NO: 23 - protein, 9 aa, Artificial Sequence
  Synthetic polypeptide
  MISC_FEATURE (2)..(2): Xaa is H or Q
  MISC_FEATURE (3)..(3): Xaa is Y or H
  MISC_FEATURE (4)..(4): Xaa is G or D
  MISC_FEATURE (6)..(6): Xaa is I or R
  MISC_FEATURE (8)..(8): Xaa is F or R
LXXXSXPXT

SEQ ID NO: 24 - protein, 9 aa, Artificial Sequence
  Synthetic polypeptide
LHYGSIPFT

SEQ ID NO: 25 - protein, 9 aa, Artificial Sequence
  Synthetic polypeptide
LQHDSRPRT

SEQ ID NO: 26 - protein, 130 aa, Artificial Sequence
  Synthetic polypeptide
  MISC_FEATURE (1)..(19): Optional leader sequence
MAVLVLLLCLVTFPNSVLTQIQLKETGPDLVQLTQTLSITCTVSGFSLTTYNVHWVRQTPGKGLEWMGAMWNGGRTDYNSAFKSRLSISRDTSKSQVFLNMNSLQADDTAKYFCARLDYWGQGVMVTVAS

SEQ ID NO: 27 - protein, 130 aa, Artificial Sequence
  Synthetic polypeptide
  MISC_FEATURE (1)..(19): Optional leader sequence
MAVLVLLLCLVTFPNFVLTQVQLKETGPDLVHLTQTLSITCTVSGFSLTTYNIHWVRQPPGKGLEWMGAMWNGGGTDYNSAFKSRLSISRDTSKSQVFLKMNSLQTDDTAKYFCATPTDWGQGVMVTVSS

SEQ ID NO: 28 - protein, 141 aa, Artificial Sequence
  Synthetic polypeptide
  MISC_FEATURE (1)..(19): Optional leader sequence
MKLWLNWIFLLTLLNGLQCEVKLLESGGGLVQPGDSMRLSCAASGFTFTDFYMNWIRQPAGKAPEWLGFIRNKVNGYTTDYNPSVKGRFTISRENTQNMLYLQMNTLRAEDTATYYCTREDYYYSGRFDYWGQGVMVTVSS

SEQ ID NO: 29 - protein, 127 aa, Artificial Sequence
  Synthetic polypeptide
  MISC_FEATURE (1)..(20): Optional leader sequence
MESHTRVFIFLLLWLSGADGETVMTQSPTSMSTSIGERVTLNCKASQSVGIDVDWYQQTPGQSPKLLIYGASNRHTGVPDRFTGSGFGRDFTLTISNVEAEDLAVYYCLHYGSIPFTFGSGTKLEIK

SEQ ID NO: 30 - protein, 127 aa, Artificial Sequence
  Synthetic polypeptide
  MISC_FEATURE (1)..(20): Optional leader seqeunce
MESHTRVFIFLLLWLSGADGETVMTQSPTSMSTSIGERVTLNCKASQSVGINVDWYQQTPGQSPKLLIYGASSRHTGVPDRFTGSGFGRDFTLTITNVEAEDLAVYYCLHYGSIPFTFGSGTRLEIK

SEQ ID NO: 31 - protein, 126 aa, Artificial Sequence
  Synthetic polypeptide
  MISC_FEATURE (1)..(19): Optional leader seqeunce
MAALQLLGLLLLWLPAMRCDIQMTQSPSFLSASVGDRVTINCKASQNINKYLDWYHQNHGEAPKLLIYHTNNLQTGIPSRFSGSGSGTDYTLTISSLQPEDVATYFCLQHDSRPRTFGGGTKLDLK

SEQ ID NO: 32 - protein, 31 aa, Artificial Sequence
  Synthetic polypeptide
KNLEPVSWSSLNPKFLSGKGLVIYPKIGDKL

SEQ ID NO: 33 - protein, 14 aa, Artificial Sequence
  Synthetic polypeptide
SWSSLNPKFLSGKG

SEQ ID NO: 34 - protein, 20 aa, Artificial Sequence
  Synthetic polypeptide
KNLEPVSWSSLNPKFLSGKG

SEQ ID NO: 35 - protein, 15 aa, Artificial Sequence
  Synthetic polypeptide
SGKGLVIYPKIGDKL

SEQ ID NO: 36 - protein, 344 aa, Artificial Sequence
  Synthetic polypeptide
MARPGQRWLGKWLVAMVVWALCRLATPLAKNLEPVSWSSLNPKFLSGKGLVIYPKIGDKLDIICPRAERPYEYYKLYLVRPEQAAACSTVLDPNVLVTCNRPEQEIRFTIKFQEFSPNYMGLEFKKHHDYYITSTSNGSLEGLENREGGVCRTRTMKIIMKVGQDPNAVTPEQLTTSRPSKEADNTVKMATQAPGSRGSLGDSDGKHETVNQEEKSGPGASGGSSGDPDGFFNSKVALFAAVGAGCVIFLLIIIFLTVLLLKLRKRHRKHTQQRAAALSLSTLASPKGGSGTAGTEPSDIIIPLRTTENNYCPHYEKVSGDYGHPVYIVQEMPPQSPANIYYKV

SEQ ID NO: 37 - protein, 235 aa, Artificial Sequence
  Synthetic polypeptide
MARPGQRWLGKWLVAMVVWALCRLATPLAKNLEPVSWSSLNPKFLSGKGLVIYPKIGDKLDIICPRAERPYEYYKLYLVRPEQAAACSTVLDPNVLVTCNRPEQEIRFTIKFQEFSPNYMGLEFKKHHDYYITSTSNGSLEGLENREGGVCRTRTMKIIMKVGQDPNAVTPEQLTTSRPSKEADNTVKMATQAPGSRGSLGDSDGKHETVNQEEKSGPGASGGSSGDPDGFFNSK

SEQ ID NO: 38 - protein, 221 aa, Artificial Sequence
  Synthetic polypeptide
MSDGDYDYLIKFLALGDSGVGKTSVLYQYTDGKFNSKFITTVGIDFREKRVVYRASGPDGATGRGQRIHLQLWDTAGQERFRSLTTAFFRDAMGFLLLFDLTNEQSFLNVRNWISQLQMHAYCENPDIVLCGNKSDLEDQRVVKEEEAIALAEKYGIPYFETSAANGTNISQAIEMLLDLIMKRMERCVDKSWIPEGVVRSNGHASTDQLSEEKEKGACGC

SEQ ID NO: 39 - protein, 218 aa, Artificial Sequence
  Synthetic polypeptide
MTDGDYDYLIKLLALGDSGVGKTTFLYRYTDNKFNPKFITTVGIDFREKRVVYNAQGPNGSSGKAFKVHLQLWDTAGQERFRSLTTAFFRDAMGFLLMFDLTSQQSFLNVRNWMSQLQANAYCENPDIVLIGNKADLPDQREVNERQARELADKYGIPYFETSAATGQNVEKAVETLLDLIMKRMEQCVEKTQIPDTVNGGNSGNLDGEKPPEKKCIC

(19) United States
(12) Patent Application Publication
(10) Pub. No.: US 2022/0213214 A1
(43) Pub. Date: Jul. 7, 2022

(54) Anti-human ephrin B1 antibodies and uses thereof

(71) Applicant: SANFORD HEALTH, Sioux Falls, SD (US)

(72) Inventor: Paola VERMEER, Sioux Falls, SD (US)

(21) Appl. No.: 17/606,847

(22) PCT Filed: May 13, 2020
(86) PCT No.: PCT/US2020/032707

§ 371 Date: Oct. 27, 2021

Related U.S. Application Data
(60) Provisional application No. 62/847,863, filed on May 14, 2019.

(51) Int. Cl. C07K 16/30

(52) U.S. Cl. CPC C07K 16/30; A61K 2039/505

(57) ABSTRACT

Disclosed herein are isolated anti-human ephrin B1 antibodies, or fragments thereof, and methods for their use in treating tumors.

### CROSS REFERENCE

This application claims priority to U.S. Provisional Patent Application Ser. No. 62/847,863 filed May 14, 2019, incorporated by reference herein in its entirety

### FEDERAL FUNDING STATEMENT

This invention was made with government support under grant number P20GM103548 awarded by the National Institute of General Medical Sciences. The government has certain rights in the invention.

### REFERENCE TO SEQUENCE LISTING

This application contains a Sequence Listing submitted as an electronic text file named “18-285-PCT_Sequence-Listing_ST25.txt”, having a size in bytes of 30 kb, and created on May 13, 2020. The information contained in this electronic file is hereby incorporated by reference in its entirety pursuant to 37 CFR § 1.52(e)(5).

### BACKGROUND

Innervated tumors are more aggressive than less innervated one. For instance, in prostate cancer, recruitment of nerve fibers to cancer tissue is associated with higher tumor proliferative indices and a higher risk of recurrence and metastasis. Denervation studies in pre-clinical and genetically engineered mouse cancer models support a functional contribution of neural elements in disease progression. These studies strongly indicate that the nervous system is not a bystander but instead an active participant in carcinogenesis and cancer progression.

### SUMMARY

In one aspect, the disclosure provides isolated anti-human ephrin B1 antibody, or fragment thereof, comprising a heavy chain that comprises 1, 2, or all 3 complementarity determining regions (CDR) selected from the group consisting of:

Heavy chain CDR1 (H-CDR1) comprising the amino acid sequence selected from the group consisting of (T/D)(Y/F)(N/Y)(V/I/M)(H/N) (SEQ ID NO:1), TYN(V/I)H (SEQ ID NO:2), TYNVH (SEQ ID NO:3), TYNIH (SEQ ID NO:4), and DFYMN (SEQ ID NO:5);

Heavy chain CDR2 (H-CDR2) comprising the amino acid sequence selected from the group consisting of (F/-)(I/-)(R/-)(A/N)(M/K)(W/V)NG(G/Y)(R/G/T)TDYN(S/P)(A/S)(F/V)K(S/G) (SEQ ID NO:6), AMWNGG(R/G)TDYNSAFKS (SEQ ID NO:7), AMWNGGRTDYNSAFKS (SEQ ID NO:8), AMWNGGGTDYNSAFKS (SEQ ID NO:9), and FIRNKVNGYTTDYNPSVKG (SEQ ID NO:10); and

Heavy chain CDR3 (H-CDR3) comprising the amino acid sequence selected from the group consisting of (E/-)(D/-)(Y/-)(Y/-)(Y/-)(S,-)(G/-)(R/-)(L/P/F)(D/I)(D/Y) (SEQ ID NO:11), LDY, PTD, and EDYYYSGRFDY (SEQ ID NO:12).

In various embodiments, the heavy chain comprises 1, 2, or all 3 of the CDRS selected from the group consisting of:

H-CDR1 comprises the amino acid sequence selected from the group consisting of TYNVH (SEQ ID NO:3), TYNIH (SEQ ID NO:4), and DFYMN (SEQ ID NO:5);

H-CDR2 comprises the amino acid sequence selected from the group consisting of AMWNGGRTDYNSAFKS (SEQ ID NO:8), AMWNGGGTDYNSAFKS (SEQ ID NO:9), and FIRNKVNGYTTDYNPSVKG (SEQ ID NO:10); and

H-CDR3 comprises the amino acid sequence selected from the group consisting of LDY, PTD, and EDYYYSGRFDY (SEQ ID NO:12); or

H-CDR1 comprises the amino acid sequence of TYNVH (SEQ ID NO:3);

H-CDR2 comprises the amino acid sequence of AMWNGGRTDYNSAFKS (SEQ ID NO:8); and

H-CDR3 comprises the amino acid sequence of LDY; or

H-CDR1 comprises the amino acid sequence of TYNIH (SEQ ID NO:4);

H-CDR2 comprises the amino acid sequence of AMWNGGGTDYNSAFKS (SEQ ID NO:9); and

H-CDR3 comprises the amino acid sequence of PTD; or

H-CDR1 comprises the amino acid sequence of DFYMN (SEQ ID NO:5);

H-CDR2 comprises the amino acid sequence of FIRNKVNGYTTDYNPSVKG (SEQ ID NO:10); and

H-CDR3 comprises the amino acid sequence of EDYYYSGRFDY (SEQ ID NO:12).

In another embodiment, the isolated anti-human ephrin B1 antibody, or fragment thereof, comprises a light chain that comprises 1, 2, or all 3 complementarity determining regions (CDR) selected from the group consisting of:

Light chain CDR1 (L-CDR1) comprises the amino acid sequence selected from the group consisting of KASQ(S/N)(V/I)(G/N)(I/K)(D/N/Y)(V/L)D (SEQ ID NO:13), KASQSVGI(D/N)VD (SEQ ID NO:14), KASQSVGIDVD (SEQ ID NO:15), KASQSVGINVD (SEQ ID NO:16), and KASQNINKYLD (SEQ ID NO:17);

Light chain CDR2 (L-CDR2) comprises the amino acid sequence selected from the group consisting of (G/H)(A/T)(S/N)(S/N)(R/L)(H/T)T (SEQ ID NO:18), GAS(N/S)RHT (SEQ ID NO:19), GASNRHT (SEQ ID NO:20), GASSRHT (SEQ ID NO:21), and HTNNLQT (SEQ ID NO:22); and

Light chain CDR3 (L-CDR3): selected from the group consisting of L(H/Q)(Y/H)(G/D)S(I/R)P(F/R)T (SEQ ID NO:23), LHYGSIPFT (SEQ ID NO:24), and LQHDSRPRT (SEQ ID NO:25).

In various embodiments, the light chain comprises 1, 2, or all 3 of the CDRS selected from the group consisting of:

L-CDR1 comprises the amino acid sequence selected from the group consisting of KASQSVGIDVD (SEQ ID NO:15), KASQSVGINVD (SEQ ID NO:16), and KASQNINKYLD (SEQ ID NO:17);

L-CDR2 comprises the amino acid sequence selected from the group consisting of GASNRHT (SEQ ID NO:20), GASSRHT (SEQ ID NO:21), and HTNNLQT (SEQ ID NO:22); and

L-CDR3 comprises the amino acid sequence selected from the group consisting of LHYGSIPFT(SEQ ID NO:24) and LQHDSRPRT (SEQ ID NO:25); or

L-CDR1 comprises the amino acid sequence of KASQSVGIDVD (SEQ ID NO:15);

L-CDR2 comprises the amino acid sequence of GASNRHT (SEQ ID NO:20); and

L-CDR3 comprises the amino acid sequence of LHYGSIPFT (SEQ ID NO:24); or

L-CDR1 comprises the amino acid sequence of KASQSVGINVD (SEQ ID NO:16);

L-CDR2 comprises the amino acid sequence of GASSRHT (SEQ ID NO:21); and

L-CDR3 comprises the amino acid sequence of LHYGSIPFT (SEQ ID NO:24); or

L-CDR1 comprises the amino acid sequence of KASQNINKYLD (SEQ ID NO:17);

L-CDR2 comprises the amino acid sequence of HTNNLQT (SEQ ID NO:22); and

L-CDR3 comprises the amino acid sequence of LQHDSRPRT (SEQ ID NO:25).

In another embodiment, the isolated anti-human ephrin B1 antibody, or fragment thereof, comprising at least 1, 2, 3, 4, 5, or all 6 complementarity determining regions (CDR) selected from the group consisting of:

Heavy chain CDR1 (H-CDR1) comprises the amino acid sequence selected from the group consisting of (T/D)(Y/F)(N/Y)(V/I/M)(H/N) (SEQ ID NO:1), TYN(V/I)H (SEQ ID NO:2), TYNVH (SEQ ID NO:3), TYNIH (SEQ ID NO:4), and DFYMN (SEQ ID NO:5);

Heavy chain CDR2 (H-CDR2) comprises the amino acid sequence selected from the group consisting of (F/-)(I/-)(R/-)(A/N)(M/K)(W/V)NG(G/Y)(R/G/T)TDYN(S/P)(A/S)(F/V)K(S/G)(SEQ ID NO:6), AMWNGG(R/G)TDYNSAFKS (SEQ ID NO:7), AMWNGGRTDYNSAFKS (SEQ ID NO:8), AMWNGGGTDYNSAFKS (SEQ ID NO:9), and FIRNKVNGYTTDYNPSVKG (SEQ ID NO:10);

Heavy chain CDR3 (H-CDR3) comprises the amino acid sequence selected from the group consisting of (E/-)(D/-)(Y/-)(Y/-)(Y/-)(S/-)(G/-)(R/-)(L/P/F)(D/T)(D/Y) (SEQ ID NO:11), LDY, PTD, and EDYYYSGRFDY (SEQ ID NO:12);

Light chain CDR1 (L-CDR1) comprises the amino acid sequence selected from the group consisting of KASQ(S/N)(V/I)(G/N)(I/K)(D/N/Y)(V/L)D (SEQ ID NO:13), KASQSVGI(D/N)VD (SEQ ID NO:14), KASQSVGIDVD (SEQ ID NO:15), KASQSVGINVD (SEQ ID NO:16), and KASQNINKYLD (SEQ ID NO:17);

Light chain CDR2 (L-CDR2) comprises the amino acid sequence selected from the group consisting of (G/H)(A/T)(S/N)(S/N)(R/L)(H/T)T (SEQ ID NO:18), GAS(N/S)RHT (SEQ ID NO:19), GASNRHT (SEQ ID NO:20). GASSRHT (SEQ ID NO:21), and HTNNLQT (SEQ ID NO:22); and

Light chain CDR3 (L-CDR3): selected from the group consisting of L(H/Q)(Y/H)(G/D)S(I/R)P(F/R)T (SEQ ID NO:23), LHYGSIPFT (SEQ ID NO:24), and LQHDSRPRT (SEQ ID NO:25).

In various embodiments, at least 1, 2, 3, 4, 5, or all 6 of the CDRS are selected from the group consisting of:

H-CDR1 comprises the amino acid sequence selected from the group consisting of TYNVH (SEQ ID NO:3), TYNIH (SEQ ID NO:4), and DFYMN (SEQ ID NO:5);

H-CDR2 comprises the amino acid sequence selected from the group consisting of AMWNGGRTDYNSAFKS (SEQ ID NO:8), AMWNGGGTDYNSAFKS (SEQ ID NO:9), and FIRNKVNGYTTDYNPSVKG (SEQ ID NO:10);

H-CDR3 comprises the amino acid sequence selected from the group consisting of LDY, PTD, and EDYYYSGRFDY (SEQ ID NO:12);

L-CDR1 comprises the amino acid sequence selected from the group consisting of KASQSVGIDVD (SEQ ID NO: 15), KASQSVGINVD (SEQ ID NO:16), and KASQNINKYLD (SEQ ID NO:17);

L-CDR2 comprises the amino acid sequence selected from the group consisting of GASNRHT (SEQ ID NO:20), GASSRHT (SEQ ID NO:21), and HTNNLQT (SEQ ID NO:22); and

L-CDR3 comprises the amino acid sequence selected from the group consisting of LHYGSIPFT(SEQ ID NO:24) and LQHDSRPRT (SEQ ID NO:25); or

H-CDR1 comprises the amino acid sequence of TYNVH (SEQ ID NO:3);

H-CDR2 comprises the amino acid sequence of AMWNGGRTDYNSAFKS (SEQ ID NO:8);

H-CDR3 comprises the amino acid sequence of LDY;

L-CDR1 comprises the amino acid sequence of KASQSVGIDVD (SEQ ID NO:15);

L-CDR2 comprises the amino acid sequence of GASNRHT (SEQ ID NO:20); and

L-CDR3 comprises the amino acid sequence of LHYGSIPFT (SEQ ID NO:24); or

H-CDR1 comprises the amino acid sequence of TYNIH (SEQ ID NO:4);

H-CDR2 comprises the amino acid sequence of AMWNGGGTDYNSAFKS (SEQ ID NO:9);

H-CDR3 comprises the amino acid sequence of PTD;

L-CDR1 comprises the amino acid sequence of KASQSVGINVD (SEQ ID NO:16);

L-CDR2 comprises the amino acid sequence of GASSRHT (SEQ ID NO:21); and

L-CDR3 comprises the amino acid sequence of LHYGSIPFT (SEQ ID NO:24); or

H-CDR1 comprises the amino acid sequence of DFYMN (SEQ ID NO:5);

H-CDR2 comprises the amino acid sequence of FIRNKVNGYTTDYNPSVKG (SEQ ID NO:10);

H-CDR3 comprises the amino acid sequence of EDYYYSGRFDY (SEQ ID NO:12);

L-CDR1 comprises the amino acid sequence of KASQNINKYLD (SEQ ID NO:17);

L-CDR2 comprises the amino acid sequence of HTNNLQT (SEQ ID NO:22); and

L-CDR3 comprises the amino acid sequence of LQHDSRPRT (SEQ ID NO:25).

In other embodiments, the anti-human ephrin B1 antibody or fragment thereof comprises a heavy chain having the amino acid sequence selected from the group consisting of the following, wherein residues in parentheses are optional:

BXD-1H7-G5
Heavy chain: Amino acids sequence (130 aa)
(SEQ ID NO: 26)
Leader sequence-FR1-CDR1-FR2-CDR2-FR3-CDR3-FR4
(MAVLVLLLCLVTFPNSVLT)QIQLKETGPDLVQLTQTLSITCTVS
GFSLTTYNVHWVRQTPGKGLEWMGAMWNGGRTDYNSAFKSRLSISR
DTSKSQVFLNMNSLQADDTAKYFCARLDYWGQGVMVTVAS
BXD-2G8-D3
Heavy chain: Amino acids sequence (130 aa)
(SEQ ID NO: 27)
Leader sequence-FR1-CDR1-FR2-CDR2-FR3-CDR3-FR4
(MAVLVLLLCLVTFPNFVLT)QVQLKETGPDLVHLTQTLSITCTVS
GFSLTTYNIHWVRQPPGKGLEWMGAMWNGGGTDYNSAFKSRLSISR
DTSKSQVFLKMNSLQTDDTAKYFCATPTDWGQGVMVTVSS
BXD-6C4-B9
Heavy chain: Amino acids sequence (141 aa)
(SEQ ID NO: 28)
Leader sequence-FR1-CDR1-FR2-CDR2-FR3-CDR3-FR4
(MKLWLNWIFLLTLLNGLQC)EVKLLESGGGLVQPGDSMRLSCAAS
GFTFTDFYMNWIRQPAGKAPEWLGFIRNKVNGYTTDYMPSVKGRFT
ISRENTQNMLYLQMNTLRAEDTATYYCTREDYYYSGRFDYWGQGVM
VTVSS

In other embodiments, the anti-human ephrin B1 antibody or fragment thereof comprises a light chain having the amino acid sequence selected from the group consisting of the following, wherein residues in parentheses are optional:

BXD-1H7-G5
Light chain: Amino acids sequence (127 aa)
Leader sequence-FR1-CDR1-FR2-CDR2-FR3-CDR3-FR4
(SEQ ID NO: 29)
(MESHTRVFIFLLLWLSGADG)ETVMTQSPTSMSTSIGERVTLNCKASQSV
GIDVDWYQQTPGQSPKLLIYGASNRHTGVPDRFTGSGFGRDFTLTISNVEA
DLAVYYCLHYGSIPFTFGSGTKLEIK
BXD-2G8-D3
Light chain: Amino acids sequence (127 aa)
Leader sequence-FR1-CDR1-FR2-CDR2-FR3-CDR3-FR4
(SEQ ID NO: 30)
(MESHTRVFIFLLLWLSGADG)ETVMTQSPTSMSTSIGERVTLNCKASQSV
GINVDWYQQTPGQSPKLLIYGASSRHTGVPDRFTGSGFGRDFTLTITNVEA
EDLAVYYCLHYGSIPFTFGSGTRLEIK
BXD-6C4-B9
Light chain: Amino acids sequence (126 aa)
Leader sequence-FR1-CDR1-FR2-CDR2-FR3-CDR3-FR4
(SEQ ID NO: 31)
(MAALQLLGLLLLWLPAMRC)DIQMTQSPSFLSASVGDRVTINCKASQNIN
KYLDWYHQNHGEAPKLLIYHTNNLQTGIPSRFSGSGSGTDYTLTISSLQPE
DVATYFCLQHDSRPRTFGGGTKLDLK

In various further embodiments, the disclosure provides anti-human ephrin B1 antibody or fragment thereof, wherein the antibody or fragment thereof binds selectively to a human ephrin B1 epitope within the amino acid sequence consisting of SEQ ID NO: 32. (KNLEPVSWSSLNPKFLSGKGLVIYPKIGDKL).

In various other embodiments, the antibody comprises a monoclonal antibody, or fragment thereof; the antibody comprises a humanized antibody, or fragment thereof; and/or the antibody or fragment thereof further comprises a detectable label and/or a therapeutic agent conjugated to the antibody or fragment thereof.

In other embodiments, the disclosure provides nucleic acids encoding an antibody of the disclosure, an expression vector comprising the nucleic acid of the disclosure operatively linked to a suitable control sequence, host cells comprising the expression vector or the nucleic acid of the disclosure, pharmaceutical compositions comprising an antibody or fragment thereof of the disclosure and a pharmaceutically acceptable carrier, and methods for treating tumors comprising administering to a subject having a tumor with an amount effective to limit tumor growth or metastasis of the anti-human ephrin B1 antibody or fragment thereof, or the pharmaceutical composition, or any embodiment or combination of embodiments disclosed herein.

### DESCRIPTION OF THE FIGURES

FIG. 1A-B. (A) To test the ability of the anti-EphrinB1 antibodies to block neurite outgrowth in vitro, they were incubated with conditioned media from SCC47-EphrinB1 cells. Twenty-four hours later, the media was used to stimulate PC12 cells. The next day, PC12 cells were fixed, stained for beta-Ill tubulin and neurite outgrowth quantified. The bar graph shows that all but one antibody (2G4) antibodies tested were able to significantly attenuate neurite outgrowth relative to that induced by conditioned media from SCC47-EphrinB1. (B) To test the utility of the EphrinB1 antibodies to block tumor growth, immune incompetent NOD SCID mice were implanted with human SCC47-EphrinB1 tumors. There were four groups of mice with N=5 mice/group. One group of mice served as the isotype matched IgG (IgG2a/2b) control group. The other three groups were each injected with a different EphrinB1 antibody clone (1H7, 2G8, or 6C4). Tumor growth was followed and mice were sacrificed at day 14 post-tumor implantation. Tumor growth of the 1H7 and 2G8 EphrinB1 antibody injected groups were significantly smaller than controls. Tumor growth of the 6C4 EphrinB1 antibody injected group showed a reduction in tumor volume, but this finding was not statistically significant.

FIG. 2A-B. NSG mice were injected with human SCC1 (HPV negative human head and neck squamous cell carcinoma cell line) cells that express endogenous (basal) EphrinB1 or SCC1 cells that stably over-express EphrinB1 (SCC1 OE #18). These animals were treated with purified antibody daily and tumor growth followed. As shown in FIG. 2A-B below, HPV negative SCC1 cells respond to antibody treatment with decreased tumor growth, whether expressing ephrin B1 at a basal level (A) or with ephrin B1 overexpressed (B).

### DETAILED DESCRIPTION

As used herein and unless otherwise indicated, the terms “a” and “an” are taken to mean “one”, “at least one” or “one or more”. Unless otherwise required by context, singular terms used herein shall include pluralities and plural terms shall include the singular.

All scientific and technical terms used in this application have meanings commonly used in the art unless otherwise specified.

As used herein, “about” means+/−5% of the recited dimension or unit.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gin; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

All embodiments of any aspect of the disclosure can be used in combination, unless the context clearly dictates otherwise.

In a first aspect, the disclosure provides isolated anti-human ephrin B1 antibodies, or ephrin B1-binding fragments thereof (referred to herein as “fragments thereof”). The anti-human ephrin B1 antibodies or fragments thereof are useful, for example, to limit tumor growth or metastasis.

In one embodiment, the ephrin B1-specific antibodies bind to an ephrin B1 protein having the sequence shown SEQ ID NO:36 below.

### Human Ephrin B1 Amino Acid Sequence

Extracellular domain bold/underlined; cytoplasmic domain italicized; transmembrane domain in enlarged/outline font

(SEQ ID NO: 36)
MARPGQRWLGKWLVAMVVWALCRLATPLAKNLEPV
SWSSLNPKFLSGKGLVIYPKIGDKLDIICPRAERP
YEYYKLYLVRPEQAAACSTVLDPNVLVTCNRPEQE
IRFTIKFQEFSPNYMGLEFKKHHDYYITSTSNGSL
EGLENREGGVCRTRTMKIIMKVGQDPNAVTPEQLT
TSRPSKEADNTVKMATQAPGSRGSLGDSDGKHETV
NQEEKSGPGASGGSSGDPDGFFNSKVALFAAVGAG
CVIFLLIIIFLTVLLLKLRKRKRKHTQQRAAALSL
STLASPKGGSGTAGTEPSDIIIPLRTTENNYCPHY
EKVSGDYGHPVYIVQEMPPQSPANIYYKV.

In another embodiment, the ephrin B1-specific antibodies bind to one or more epitopes in the extracellular domain (ECD) of an ephrin B1 protein, where the ECD sequence comprises or consists of the sequence shown as SEQ ID NO:37 below:

(SEQ ID NO: 37; human ephrin B1
extracellular domain)
MARPGQRWLGKWLVAMVVWALCRLATPLAKNLEPV
SWSSLNPKFLSGKGLVIYPKIGDKLDIICFRAERP
YEYYKLYLVRPEQAAACSTVLDPNVLVTCNRPEQE
IRFTIKFQEFSPNYMGLEFKKHHDYYITSTSNGSL
EGLENREGGVCRTRTMKIIMKVGQDPNAVTPEQLT
TSRPSKEADNTVKMATQAPGSRGSLGDSDGKHETV
NQEEKSGPGASGGSSGDPDGFFNSK.

In another embodiment the ephrin B1-specific antibodies bind to an ephrin B1 protein having the amino acid sequence of SEQ ID NO:36 but which have one or more of the following amino acid changes relative to the amino acid sequence of SEQ ID NO:36:

### Position 27 P to R

### Position 54 P to L

### Position 62 I to T

### Position 98 L to S

### Position 111 T to I

### Position 115 Q to P

### Position 119 P to T

### Position 119 P to S

### Position 137 T to A

### Position 138 S to F

### Position 151 G to S

### Position 151 G to V

### Position 153 C to S

### Position 153 C to Y

### Position 155 T to P

### Position 158 M to I

### Position 158 M to V

### Position 182 S to R

These positional changes are present in variants of the human EphrinB1 protein (SEQ ID NO:36), such as variants associated with craniofrontonasal syndrome.

As disclosed herein, “antibody” refers to immunoglobulin molecules and immunologically active portions of immunoglobulin molecules, i.e., molecules that contain an antigen binding site that immunospecifically binds an epitope in human EphrinB1 protein. As such, the term antibody encompasses not only whole antibody molecules, but also antibody fragments as well as variants (including derivatives) of antibodies and antibody fragments. Such antibody or antibody fragments thereof may include, but are not limited to monoclonal antibodies, humanized antibodies, chimeric antibodies, Fab′, F(ab′)₂, Fab, Fv, rIgG, recombinant single chain Fv fragments (scFv), bivalent or bispecific molecules, diabodies, triabodies, and tetrabodies.

As used herein, “isolated” means that the indicated molecule is present in the substantial absence of other biological macromolecules of the same type. The term “isolated” as used herein preferably means at least 75% by weight, more preferably at least 85% by weight, more preferably still at least 95% by weight, and most preferably at least 98% by weight, of biological macromolecules of the same type are present.

In one embodiment, the anti-human ephrin B1 antibody, or fragment thereof, comprises a heavy chain that comprises 1, 2, or all 3 complementarity determining regions (CDR) selected from the group consisting of:

Heavy chain CDR1 (H-CDR1) comprising the amino acid sequence selected from the group consisting of (T/D)(Y/F)(N/Y)(V/I/M)(H/N) (SEQ ID NO:1), TYN(V/I)H (SEQ ID NO:2), TYNVH (SEQ ID NO:3), TYNIH (SEQ ID NO:4), and DFYMN (SEQ ID NO:5);

Heavy chain CDR2 (H-CDR2) comprising the amino acid sequence selected from the group consisting of (F/-)(I-)(R/-)(A/N)(M/K)(W/V)NG(G/Y)(R/G/T)TDYN(S/P)(A/S)(F/V)K(S/G) (SEQ ID NO:6), AMWNGG(R/G)TDYNSAFKS (SEQ ID NO:7), AMWNGGRTDYNSAFKS (SEQ ID NO:8), AMWNGGGTDYNSAFKS (SEQ ID NO:9), and FIRNKVNGYTTDYNPSVKG (SEQ ID NO:10); and

Heavy chain CDR3 (H-CDR3) comprising the amino acid sequence selected from the group consisting of (E/-)(D/-)(Y/-)(Y/-)(Y/-)(S/-)(G/-)(R/-)(L/P/F)(D/T)(D/Y) (SEQ ID NO:11), LDY, PTD, and EDYYYSGRFDY (SEQ ID NO:12).

In another embodiment, the heavy chain comprises 1, 2, or all 3 of the CDRS selected from the group consisting of:

H-CDR1 comprises the amino acid sequence selected from the group consisting of TYNVH (SEQ ID NO:3), TYNIH (SEQ ID NO:4), and DFYMN (SEQ ID NO:5);

H-CDR2 comprises the amino acid sequence selected from the group consisting of AMWNGGRTDYNSAFKS (SEQ ID NO:8), AMWNGGGTDYNSAFKS (SEQ ID NO:9), and FIRNKVNGYTTDYNPSVKG (SEQ ID NO:10); and

H-CDR3 comprises the amino acid sequence selected from the group consisting of LDY, PTD, and EDYYYSGRFDY (SEQ ID NO:12).

In a further embodiment, the heavy chain comprises 1, 2, or all 3 of the CDRS as follows:

H-CDR1 comprises the amino acid sequence of TYNVH (SEQ ID NO:3);

H-CDR2 comprises the amino acid sequence of AMWNGGRTDYNSAFKS (SEQ ID NO:8); and

H-CDR3 comprises the amino acid sequence of LDY.

In one embodiment, the heavy chain comprises 1, 2, or all 3 of the CDRS as follows:

H-CDR1 comprises the amino acid sequence of TYNIH (SEQ ID NO:4);

H-CDR2 comprises the amino acid sequence of AMWNGGGTDYNSAFKS (SEQ ID NO:9); and

H-CDR3 comprises the amino acid sequence of PTD.

In another embodiment, the heavy chain comprises 1, 2, or all 3 of the CDRS as follows:

H-CDR1 comprises the amino acid sequence of DFYMN (SEQ ID NO:5);

H-CDR2 comprises the amino acid sequence of FIRNKVNGYTTDYNPSVKG (SEQ ID NO:10); and

H-CDR3 comprises the amino acid sequence of EDYYYSGRFDY (SEQ ID NO:12).

In another embodiment, the disclosure provides isolated anti-human ephrin B1 antibodies, or fragment thereof, comprising a light chain that comprises 1, 2, or all 3 complementarity determining regions (CDR) selected from the group consisting of:

Light chain CDR1 (L-CDR1) comprises the amino acid sequence selected from the group consisting of KASQ(S/N)(V/I)(G/N)(I/K)(D/N/Y)(V/L)D (SEQ ID NO:13), KASQSVGI(D/N)VD (SEQ ID NO:14), KASQSVGIDVD (SEQ ID NO:15), KASQSVGINVD (SEQ ID NO:16), and KASQNINKYLD (SEQ ID NO:17);

Light chain CDR2 (L-CDR2) comprises the amino acid sequence selected from the group consisting of (G/H)(A/T)(S/N)(S/N)(R/L)(H/T)T (SEQ ID NO:18), GAS(N/S)RHT (SEQ ID NO:19), GASNRHT (SEQ ID NO:20), GASSRHT (SEQ ID NO:21), and HTNNLQT (SEQ ID NO:22); and

Light chain CDR3 (L-CDR3): selected from the group consisting of L(H/Q)(Y/H)(G/D)S(I/R)P(F/R)T (SEQ ID NO:23), LHYGSIPFT (SEQ ID NO:24), and LQHDSRPRT (SEQ ID NO:25).

In one embodiment, the light chain comprises 1, 2, or all 3 of the CDRS selected from the group consisting of:

L-CDR1 comprises the amino acid sequence selected from the group consisting of KASQSVGIDVD (SEQ ID NO:15), KASQSVGINVD (SEQ ID NO:16), and KASQNINKYLD (SEQ ID NO:17);

L-CDR2 comprises the amino acid sequence selected from the group consisting of GASNRHT (SEQ ID NO:20), GASSRHT (SEQ ID NO:21), and HTNNLQT (SEQ ID NO:22); and

L-CDR3 comprises the amino acid sequence selected from the group consisting of LHYGSIPFT(SEQ ID NO:24) and LQHDSRPRT (SEQ ID NO:25).

In another embodiment, the light chain comprises 1, 2, or all 3 of the CDRS are as follows:

L-CDR1 comprises the amino acid sequence of KASQSVGIDVD (SEQ ID NO:15);

L-CDR2 comprises the amino acid sequence of GASNRHT (SEQ ID NO:20); and

L-CDR3 comprises the amino acid sequence of LHYGSIPFT (SEQ ID NO:24).

In a further embodiment, the light chain comprises 1, 2, or all 3 of the CDRS are as follows:

L-CDR1 comprises the amino acid sequence of KASQSVGINVD (SEQ ID NO:16);

L-CDR2 comprises the amino acid sequence of GASSRHT (SEQ ID NO:21); and

L-CDR3 comprises the amino acid sequence of LHYGSIPFT (SEQ ID NO:24).

In one embodiment, the light chain comprises 1, 2, or all 3 of the CDRS are as follows:

L-CDR1 comprises the amino acid sequence of KASQNINKYLD (SEQ ID NO:17);

L-CDR2 comprises the amino acid sequence of HTNNLQT (SEQ ID NO:22); and

L-CDR3 comprises the amino acid sequence of LQHDSRPRT (SEQ ID NO:25).

In another embodiment, the isolated anti-human ephrin B1 antibodies, or fragments thereof, comprise at least 1, 2, 3, 4, 5, or all 6 complementarity determining regions (CDR) selected from the group consisting of:

Heavy chain CDR1 (H-CDR1) comprising the amino acid sequence selected from the group consisting of (T/D)(Y/F)(N/Y)(V/I/M)(H/N) (SEQ ID NO:1). TYN(V/I)H (SEQ ID NO:2), TYNVH (SEQ ID NO:3), TYNIH (SEQ ID NO:4), and DFYMN (SEQ ID NO:5);

Heavy chain CDR2 (H-CDR2) comprising the amino acid sequence selected from the group consisting of (F/-)(I/-)(R/-)(A/N)(M/K)(W/V)NG(G/Y)(R/G/T)TDYN(S/P)(A/S)(F/V)K(S/G) (SEQ ID NO:6), AMWNGG(R/G)TDYNSAFKS (SEQ ID NO:7), AMWNGGRTDYNSAFKS (SEQ ID NO:8), AMWNGGGTDYNSAFKS (SEQ ID NO:9), and FIRNKVNGYTTDYNPSVKG (SEQ ID NO:10);

Heavy chain CDR3 (H-CDR3) comprising the amino acid sequence selected from the group consisting of (E/-)(D/-)(Y/-)(Y/-)(Y/-)(S/-)(G/-)(R/-)(L/P/F)(D/T)(D/Y) (SEQ ID NO:11), LDY, PTD, and EDYYYSGRFDY (SEQ ID NO:12);

Light chain CDR1 (L-CDR1) comprising the amino acid sequence selected from the group consisting of KASQ(S/N)(V/I)(G/N)(I/K)(D/N/Y)(V/L)D (SEQ ID NO:13), KASQSVGI(D/N)VD (SEQ ID NO:14), KASQSVGIDVD (SEQ ID NO:15), KASQSVGINVD (SEQ ID NO:16), and KASQNINKYLD (SEQ ID NO:17);

Light chain CDR2 (L-CDR2) comprising the amino acid sequence selected from the group consisting of (G/H)(A/T)(S/N)(S/N)(R/L)(H/T)T (SEQ ID NO:18), GAS(N/S)RHT (SEQ ID NO:19), GASNRHT (SEQ ID NO:20), GASSRHT (SEQ ID NO:21), and HTNNLQT (SEQ ID NO:22); and

Light chain CDR3 (L-CDR3): comprising the amino acid sequence selected from the group consisting of L(H/Q)(Y/H)(G/D)S(I/R)P(F/R)T (SEQ ID NO:23), LHYGSIPFT (SEQ ID NO:24), and LQHDSRPRT (SEQ ID NO:25).

In a further embodiment, at least 1, 2, 3, 4, 5, or all 6 of the CDRS are selected from the group consisting of:

H-CDR1 comprising the amino acid sequence selected from the group consisting of TYNVH (SEQ ID NO:3), TYNIH (SEQ ID NO:4), and DFYMN (SEQ ID NO:5);

H-CDR2 comprising the amino acid sequence selected from the group consisting of AMWNGGRTDYNSAFKS (SEQ ID NO:8), AMWNGGGTDYNSAFKS (SEQ ID NO:9), and FIRNKVNGYTTDYNPSVKG (SEQ ID NO:10);

H-CDR3 comprising the amino acid sequence selected from the group consisting of LDY, PTD, and EDYYYSGRFDY (SEQ ID NO:12);

L-CDR1 comprising the amino acid sequence selected from the group consisting of KASQSVGIDVD (SEQ ID NO:15), KASQSVGINVD (SEQ ID NO:16), and KASQNINKYLD (SEQ ID NO:17);

L-CDR2 comprising the amino acid sequence selected from the group consisting of GASNRHT (SEQ ID NO:20), GASSRHT (SEQ ID NO:21), and HTNNLQT (SEQ ID NO:22); and

L-CDR3 comprising the amino acid sequence selected from the group consisting of LHYGSIPFT(SEQ ID NO:24) and LQHDSRPRT (SEQ ID NO:25).

In another embodiment, at least 1, 2, 3, 4, 5, or all 6 of the CDRS are as follows:

H-CDR1 comprises the amino acid sequence of TYNVH (SEQ ID NO:3);

H-CDR2 comprises the amino acid sequence of AMWNGGRTDYNSAFKS (SEQ ID NO:8);

H-CDR3 comprises the amino acid sequence of LDY;

L-CDR1 comprises the amino acid sequence of KASQSVGIDVD (SEQ ID NO:15);

L-CDR2 comprises the amino acid sequence of GASNRHT (SEQ ID NO:20); and

L-CDR3 comprises the amino acid sequence of LHYGSIPFT (SEQ ID NO:24).

In one embodiment, at least 1, 2, 3, 4, 5, or all 6 of the CDRS are as follows:

H-CDR1 comprises the amino acid sequence of TYNIH (SEQ ID NO:4);

H-CDR2 comprises the amino acid sequence of AMWNGGGTDYNSAFKS (SEQ ID NO:9);

H-CDR3 comprises the amino acid sequence of PTD;

L-CDR1 comprises the amino acid sequence of KASQSVGINVD (SEQ ID NO:16);

L-CDR2 comprises the amino acid sequence of GASSRHT (SEQ ID NO:21); and

L-CDR3 comprises the amino acid sequence of LHYGSIPFT (SEQ ID NO:24).

In a further embodiment, at least 1, 2, 3, 4, 5, or all 6 of the CDRS are as follows:

H-CDR1 comprises the amino acid sequence of DFYMN (SEQ ID NO:5);

H-CDR2 comprises the amino acid sequence of FIRNKVNGYTTDYNPSVKG (SEQ ID NO:10);

H-CDR3 comprises the amino acid sequence of EDYYYSGRFDY (SEQ ID NO:12);

L-CDR1 comprises the amino acid sequence of KASQNINKYLD (SEQ ID NO:17);

L-CDR2 comprises the amino acid sequence of HTNNLQT (SEQ ID NO:22); and

L-CDR3 comprises the amino acid sequence of LQHDSRPRT (SEQ ID NO:25).

In one embodiment, the anti-human ephrin B1 antibody or fragment thereof comprises a heavy chain having the amino acid sequence selected from the group consisting of the following, wherein residues in parentheses are optional:

BXD-1H7-G5
Heavy chain: Amino acids sequence (130 aa)
Leader sequence-FR1-CDR1-FR2-CDR2-FR3-CDR3-FR4
(SEQ ID NO: 26)
(MAVLVLLLCLVTFPNSVLT)QIQLKETGPDLVQLTQTLSITCTVSGESLTTYNVHWVRQTPGKGLE
WMGAMWNGGRTDYNSAFKSRLSISRDTSKSQVFLNMNSLQADDTAKYFCARLDYWGQGVMVTVAS
BXD-2G13-D3
Heavy chain: Amino acids sequence (130 aa)
Leader sequence-FR1-CDR1-FR2-CDR2-FR3-CDR3-FR4
(SEQ ID NO: 27)
(MAVLVLLLCLVTFPNFVLT)QVQLKETGPDLVHLTQTLSITCTVSGFSLTTYNIHWVRQPPGKG
LEWMGAMWNGGGTDYNSAFKSRLSISRDTSKSQVFLKMNSLQTDDTAKYFCATPTDWGQGVMVTV
SS
BXD-6C4-B9
Heavy chain: Amino acids sequence (141 aa)
Leader sequence-FR1-CDR1-FR2-CDR2-FR3-CDR3-FR4
(SEQ ID NO: 28)
(MKLWLNWIFLLTLLNGLQC)EVKLLESGGGLVQPGDSMRLSCAASGFTFTDFYMNWIRQPAGKA
PEWLGFIRNKVNGYTTDYMPSVKGRFTISRENTQNMLYLQMNTLRAEDTATYYCTREDYYYSGRF
DYWGQGVMVTVSS

In another embodiment, the anti-human ephrin B1 antibody or fragment thereof comprises a light chain having the amino acid sequence selected from the group consisting of the following, wherein residues in parentheses are optional:

BXD-1H7-G5
Light chain: Amino acids sequence (127 aa)
Leader sequence-FR1-CDR1-FR2-CDR2-FR3-CDR3-FR4
(SEQ ID NO: 29)
(MESHTRVFIFLLLWLSGADG)ETVMTQSPTSMSTSIGERVTLNCKASQSVGIDVDWYQQTPGQSPK
LLIYGASNRHTGVPDRFTGSGFGRDFTLTISNVEAEDLAVYYCLHYGSIPFTFGSGTKLEIK
BXD-2G8-D3
Light chain: Amino acids sequence (127 aa)
Leader sequence-FR1-CDR1-FR2-CDR2-FR3-CDR3-FR4
(SEQ ID NO: 30)
(MESHTRVFIFLLLWLSGADG)ETVMTQSPTSMSTSIGERVTLNCKASQSVGINVDWYQQTPGQS
PKLLIYGASSRHTGVPDRFTGSGFGRDFTLTITNVEAEDLAVYYCLHYGSIPFTFGSGTRLEIK
BXD-6C4-B9
Light chain: Amino acids sequence (126 aa)
Leader sequence-FR1-CDR1-FR2-CDR2-FR3-CDR3-FR4
(SEQ ID NO: 31)
(MAALQLLGLLLLWLPAMRC)DIQMTQSPSFLSASVGDRVTINCKASQNINKYLDWYHQNHGEAP
KLLIYHTNNLQTGIPSRFSGSGSGTDYTLTISSLQPEDVATYFCLQHDSRPRTFGGGTKLDLK

In a further embodiment, the anti-human ephrin B1 antibody or fragment thereof comprises a heavy chain having the amino acid sequence below, wherein residues in parentheses are optional:

BXD-1H7-G5
Heavy chain: Amino acids sequence (130 aa)
Leader sequence-FR1-CDR1-FR2-CDR2-FR3-CDR3-FR4
(SEQ ID NO: 26)
(MAVLVLLLCLVTFPNSVLT)QIQLKETGEOLVQLTQTLSITCTVSGFSLTTYNVHWVRQTPGKGLE
WMGAMWNGGRTDYNSAFKSRLSISRDTSKSQVFLNMNSLQADDTAKYFCARLDYWGQGVMVTVAS;
and
a light chain having the amino acid sequence below, wherein residues in
parentheses are optional:
BXD-1H7-G5
Light chain: Amino acids sequence (127 aa)
Leader sequence-FR1-CDR1-FR2-CDR2-FR3-CDR3-FR4
(SEQ ID NO: 29)
(MESHTRVFIFLLLWLSGADG)ETVMTQSPTSMSTSIGERVTLNCKASOSVGIDVDWYQQTPGQSPK
LLIYGASNRHTGVPDRFTGSGFGRDFTLTISNVEAEDLAVYYCLHYGSIPFTFGSGTKLEIK.

In one embodiment, the anti-human ephrin B1 antibody or fragment thereof comprises a heavy chain having the amino acid sequence below, wherein residues in parentheses are optional:

BXD-2G8-D3
Heavy chain: Amino acids sequence (130 aa)
Leader sequence-FR1-CDR1-FR2-CDR2-FR3-CDR3-FR4
(SEQ ID NO: 27)
(MAVLVLLLCLVTFPNFVLT)QVQLKETGPDLVHLTQTLSITCTVSGFSLT
TYNIHWVRQPPGKGLEWMGAMWNGGGTDYNSAFKSRLSISRDTSKSQVFLK
MNSLQTDDTAKYFCATPTDWGQGVMVTVSS;
and

a light chain having the amino acid sequence below, wherein residues in parentheses are optional:

BXD-2G8-D3
Light chain: Amino acids sequence (127 aa)
Leader sequence-FR1-CDR1-FR2-CDR2-FR3-CDR3-FR4
(SEQ ID NO: 30)
(MESHTRVFIFLLLWLSGADG)ETVMTQSPTSMSTSIGERVTLNCKASQSVGINVDWYQQTPGQS
PKLLIYGASSRHTGVPDRFTGSGEGRDFTLTITNVEAEDLAVYYCLHYGSIPFTFGSGTRLEIK.

In another embodiment, the anti-human ephrin B1 antibody or fragment thereof comprises a heavy chain having the amino acid sequence below, wherein residues in parentheses are optional:

BXD-6C4-B9
Heavy chain: Amino acids sequence (141 aa)
Leader sequence-FR1-CDR1-FR2-CDR2-FR3-CDR3-FR4
(SEQ ID NO: 28)
(MKLWLNWIFLLTLLNGLQC)EVKLLESGGGLVQPGDSMRLSCAASGETFTDFYMNWIRQPAGKA
PEWLGFIRNKVNGYTTDYMPSVKGRFTISRENTQNMLYLQMNTLRAEDTATYYCTREDYYYSGRF
DYWGQGVMVTVSS;
and

a light chain having the amino acid sequence below, wherein residues in parentheses are optional:

BXD-6C4-B9
Light chain: Amino acids sequence (126 aa)
Leader sequence-FR1-CDR1-FR2-CDR2-FR3-CDR3-FR4
(SEQ ID NO: 31)
(MAALQLLGLLLLWLPAMRC)DIQMTQSPSFLSASVGDRVTINCKASQNINKYLDWYHQNHGEAP
KLLIYHTNNLQTGIPSRFSGSGSGTDYTLTISSLQPEDVATYFCLQHDSRPRTFGGGTKLDKK.

In one embodiment of all of the above, optional amino acid residues are absent. In another embodiment, optional amino acid residues are present.

In another embodiment, the anti-human ephrin B1 antibody or fragment thereof binds to a human ephrin B1 epitope within the amino acid sequence consisting of SEQ ID NO: 32 (KNLEPVSWSSLNPKFLSGKGLVIYPKIGDKL). In one embodiment, the antibody or fragment thereof binds to a human ephrin B1 epitope of at least 14 amino acids in length within the amino acid sequence consisting of SEQ ID NO: 32 (KNLEPVSWSSLNPKFLSGKGLVIYPKIGDKL). In another embodiment, the antibody or fragment thereof binds to a human ephrin B1 epitope of between 14 amino acids and 20 amino acids in length within the amino acid sequence consisting of SEQ ID NO: 32 (KNLEPVSWSSLNPKFLSGKGLVIYPKIGDKL). In a further embodiment, the human ephrin B1 epitope consists of the amino acid sequence selected from the group consisting of SEQ ID NOS:33-35 SWSSLNPKFLSGKG (SEQ ID NO:33); KNLEPVSWSSLNPKFLSGKG (SEQ ID NO:34); and SGKGLVIYPKIGDKL (SEQ ID NO:35).

In another aspect, the disclosure provides antibodies and fragments thereof that selectively bind to the same epitope as any of the anti-human ephrin B1 antibodies or antigen-binding fragments thereof of the present disclosure. In one embodiment, the anti-human ephrin B1 antibodies or fragments bind selectively to a human ephrin B1 epitope within the amino acid sequence consisting of SEQ ID NO: 32 (KNLEPVSWSSLNPKFLSGKGLVIYPKIGDKL). In one embodiment, the human ephrin B1 epitope is at least 14 amino acids in length within the amino acid sequence consisting of SEQ ID NO: 32 (KNLEPVSWSSLNPKFLSGKGLVIYPKIGDKL). In another embodiment, the human ephrin B1 epitope is between 14 amino acids and 20 amino acids in length within the amino acid sequence consisting of SEQ ID NO: 32 (KNLEPVSWSSLNPKFLSGKGLVIYPKIGDKL). In a further embodiment, the human ephrin B1 epitope consists of the amino acid sequence selected from the group consisting of SEQ ID NOS:33-35.

The anti-human ephrin B1 antibodies or fragments thereof may be linked with any additional component as deemed suitable for an intended use. In one embodiment, the anti-human ephrin B1 antibody or fragment thereof further comprises a detectable label. Such embodiments may be useful for monitoring a course of treatment, for example. Any suitable detectable label may be bound, either covalently, genetically, or by any other means, to the antibody, including but not limited to radioactive isotopes, fluorescent molecules, magnetic particles (including nanoparticles), metal particles (including nanoparticles), phosphorescent molecules, and enzymes.

In other embodiments, the anti-human ephrin B1 antibody or fragment thereof may further comprise a therapeutic agent conjugated to the antibody or fragment thereof. Any suitable additional therapeutic agent for a given purpose may be linked, including but not limited to an inhibitor of tumor exosomal release (including but not limited to an inhibitor of Rab27a, an inhibitor of Rab27b, and/or GW4869, or pharmaceutical salts thereof); an inhibitor of the interaction between E6 and PTPN13 (including but not limited to peptides that compete with E6 for binding to PTPN13, or that compete with PTPN13 for binding to E6); and/or inhibitors of ephrin B1 phosphorylation (including but not limited to Src kinase inhibitors, including but not limited to dasatanib or a pharmaceutically acceptable salt thereof). The structure of GW4869 is provided below, and its use for exosomal release is provided in Chen et al., Journal of Cell Commun Signal 2018 12: 343-357 PMID 29063370; Richards et al. Oncogene 2017 36: 1770-1778, PMID 27669441; Essandoh et al., Biochim Biophys Acta 2015 1852: 2362-71 PMID 26300484; and Gong et al., Oncotarget 2017 8: 45200-45212 PMID 28423355.

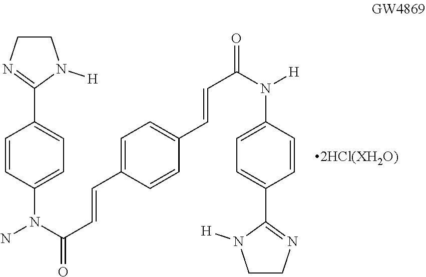 [SMILES: NN(C(=O)/C=C/c1ccc(/C=C/C(=O)Nc2ccc(C3=NCCN3)cc2)cc1)c1ccc(C2=NCCN2)cc1]

In another aspect isolated nucleic acids are disclosed encoding the antibody of any embodiment or combination of embodiments disclosed herein. The isolated nucleic acid sequence may comprise RNA or DNA. Such isolated nucleic acid sequences may comprise additional sequences useful for promoting expression and/or purification of the encoded protein, including but not limited to polyA sequences, modified Kozak sequences, and sequences encoding epitope tags, export signals, and secretory signals, nuclear localization signals, and plasma membrane localization signals.

In a further aspect recombinant expression vectors comprising the isolated nucleic acid of the disclosure are provided. “Recombinant expression vector” includes vectors that operatively link a nucleic acid coding region or gene to any promoter capable of effecting expression of the gene product. The promoter sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector is replicable in a suitable host organism either as an episome or by integration into host chromosomal DNA. In various embodiments, the expression vector comprises a plasmid or viral vector.

In a further aspect recombinant host cells comprising the nucleic acid vector of the disclosure are provided. The host cells can be either prokaryotic or eukaryotic. The cells can be transiently or stably transfected. In one embodiment, the cells are hybridoma cells that express and secrete antibodies of the present disclosure. Thus, the recombinant host cells can be used, for example in methods for producing antibody, comprising:

(a) culturing the recombinant host cell under conditions suitable for expression of the nucleic-acid encoded antibody; and

(b) isolating the antibody from the cultured cells.

Suitable conditions for expression of the nucleic-acid encoded antibody can be determined by those of skill in the art based on the teachings herein, the specific host cells and vectors used, and the general knowledge of those of skill in the art.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, e.g., recombinant cells express genes that are not found within the native (non-recombinant) form of the cell or express native genes that are otherwise abnormally expressed, under expressed or not expressed at all. By the term “recombinant nucleic acid” herein is meant nucleic acid, originally formed in vitro, in general, by the manipulation of nucleic acid, e.g., using polymerases and endonucleases, in a form not normally found in nature. In this manner, operably linkage of different sequences is achieved. Thus an isolated nucleic acid, in a linear form, or an expression vector formed in vitro by ligating DNA molecules that are not normally joined, are both considered recombinant for the purposes disclosed herein. It is understood that once a recombinant nucleic acid is made and reintroduced into a host cell or organism, it will replicate non-recombinantly, i.e., using the in vivo cellular machinery of the host cell rather than in vitro manipulations; however, such nucleic acids, once produced recombinantly, although subsequently replicated non-recombinantly, are still considered recombinant for the purposes disclosed herein.

In one aspect pharmaceutical compositions are provided, comprising:

(a) the antibody, isolated nucleic acid, recombinant expression vector, or host cell of any embodiment or combination of embodiments disclosed herein; and

(b) a pharmaceutically acceptable carrier.

For example, the antibodies of the disclosure may be present in a pharmaceutical formulation. In this embodiment, the antibodies are combined with a pharmaceutically acceptable carrier. Suitable acids which are capable of forming such salts include inorganic acids such as hydrochloric acid, hydrobromic acid, perchloric acid, nitric acid, thiocyanic acid, sulfuric acid, phosphoric acid and the like; and organic acids such as formic acid, acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid and the like. Suitable bases capable of forming such salts include inorganic bases such as sodium hydroxide, ammonium hydroxide, potassium hydroxide and the like; and organic bases such as mono-, di- and tri-alkyl and aryl amines (e.g., triethylamine, diisopropyl amine, methyl amine, dimethyl amine and the like) and optionally substituted ethanol-amines (e.g., ethanolamine, diethanolamine and the like).

The pharmaceutical composition may comprise in addition to the composition of the invention (a) a lyoprotectant; (b) a surfactant; (c) a bulking agent; (d) a tonicity adjusting agent; (e) a stabilizer; (f) a preservative and/or (g) a buffer. In some embodiments, the buffer in the pharmaceutical composition is a Tris buffer, a histidine buffer, a phosphate buffer, a citrate buffer or an acetate buffer. The pharmaceutical composition may also include a lyoprotectant, e.g. sucrose, sorbitol or trehalose. In certain embodiments, the pharmaceutical composition includes a preservative e.g. benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other embodiments, the pharmaceutical composition includes a bulking agent, like glycine. In yet other embodiments, the pharmaceutical composition includes a surfactant e.g., polysorbate-20, polysorbate-40, polysorbate-60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The pharmaceutical composition may also include a tonicity adjusting agent, e.g., a compound that renders the formulation substantially isotonic or isoosmotic with human blood. Exemplary tonicity adjusting agents include sucrose, sorbitol, glycine, methionine, mannitol, dextrose, inositol, sodium chloride, arginine and arginine hydrochloride. In other embodiments, the pharmaceutical composition additionally includes a stabilizer, e.g., a molecule which, when combined with a protein of interest substantially prevents or reduces chemical and/or physical instability of the protein of interest in lyophilized or liquid form. Exemplary stabilizers include sucrose, sorbitol, glycine, inositol, sodium chloride, methionine, arginine, and arginine hydrochloride.

The pharmaceutical compositions of the invention may be made up in any suitable formulation, preferably in formulations suitable for administration by injection. Such pharmaceutical compositions can be used, for example, in the therapeutic methods disclosed herein.

The pharmaceutical compositions may contain any other components as deemed appropriate for a given use, such as additional therapeutics. In one embodiment, the pharmaceutical compositions further comprise an inhibitor of tumor exosomal release, or a pharmaceutically acceptable salt thereof. In one embodiment, the inhibitor of tumor exosomal release comprises an inhibitor of Rab27a and/or an inhibitor of Rab27b. In another embodiment,

the inhibitor of Rab27a and/or the inhibitor of Rab27b are selected from the group consisting Rab27a and/or Rab27b-specific antibodies, aptamers, small interfering RNAs, small internally segmented interfering RNAs, short hairpin RNAs, microRNAs, and/or antisense oligonucleotides.

In another aspect are provided methods for tumor treatment comprising administering to a subject having a tumor an amount effective to limit tumor growth or metastasis of the anti-human ephrin B1 antibody or fragment thereof, or the pharmaceutical composition, of any embodiment or combination of embodiments disclosed herein. As shown in the examples that follow, the inventors have demonstrated that the antibodies of the disclosure are useful to limit tumor growth or metastasis.

As used here, the terms “treating tumor growth” means (i) limiting tumor size, (ii) limiting the rate of increase in tumor size, (iii) reducing tumor innervation, (iv) limiting the rate of increase in tumor innervation, (v) limiting tumor metastases, (vi) limiting the rate of increase in tumor metastases, (vii) limiting side effects caused by tumors (i.e., pain, sickness behavior, etc.), and/or (viii) limiting the rate of increase of side effects caused by tumors.

The amount effective of the anti-human ephrin B1 antibody or fragment thereof to be administered is any amount that will achieve the goal of treating the tumor, and can be determined by one of skill in the art (such as an attending physician) in light of all circumstances, including but not limited to the type of tumor, the subject's condition, other therapeutic treatments that the subject is undergoing (i.e.: chemotherapy, radiation therapy, surgery to remove the tumor, etc.), and all other contributing factors.

As used herein, the term “subject” or “patient” is meant any subject for which therapy is desired, including humans, cattle, dogs, cats, guinea pigs, rabbits, rats, mice, horses, chickens, and so on. Most preferably, the subject is human.

In one embodiment, the methods serve to limit tumor innervation. As used herein, “tumor innervation” is defined as neural fibers invading in, around and/or through a tumor mass. As used herein, “limiting innervation” is defined to include any reduction (i.e., 1%, 2%, 5%, 10%, 15%, 20%, 25%, 30%, 35%, 40%, 50%, or greater reduction) in neo-innervation or existing innervation, compared to no treatment with the anti-human ephrin B1 antibody or fragment thereof.

In one embodiment, the methods of the disclosure further comprise administering to the subject an amount effective of an inhibitor of tumor exosomal release. Any suitable inhibitor of tumor exosomal release may be used, including but not limited to inhibitors of Rab27a and/or Rab27b. Rab27a and Rab27b are members of the small GTPase Rab family that functions in the release of exosomes. In this embodiment, the inhibitor of Rab27a and/or the inhibitor of Rab27b include, but are not limited to Rab27a and/or the Rab27b-specific antibodies, aptamers, small interfering RNAs, small internally segmented interfering RNAs, short hairpin RNAs, microRNAs, antisense oligonucleotides, and/or small molecule inhibitors. The amino acid sequence of human Rab27a and Rab27b are provided below.

Human Rab27a:
(SEQ ID NO: 38)
MSDGDYDYLIKFLALGDSGVGKTSVLYQYTDGKFN
SKFITTVGIDFREKRVVYRASGPDGATGRGQRIHL
QLWDTAGQERFRSLTTAFFRDAMGFLLLFDLTNEQ
SFLNVRNWISQLQMHAYCENPDIVLCGNKSDLEDQ
RVVKEEEAIALAEKYGIPYFETSAANGTNISQAIE
MLLDLIMKRMERCVDKSWIPEGVVRSNGHASTDQL
SEEKEKGACGC
Human Rab27b:
(SEQ ID NO: 39)
MTDGDYDYLIKLLALGDSGVGKTTFLYRYTDNKFN
PKFITTVGIDFREKRVVYNAQGFNGSSGKAFKVHL
QLWDTAGQERFR8LTTAFFRDAMGFLLMFDLTSQQ
SFLNVRNWMSQLQANAYCENPDIVLIGNKADLPDQ
REVNERQARELADKYGIPYFETSAATGQNVEKAVE
TLLDLIMKRMEQCVEKTQIFDTVNGGNSGNLDGEK
PPEKKCIC

The methods of the disclosure may be used to treat any suitable tumor type. In one embodiment, the tumor may be any innervated solid tumor. In various non-limiting embodiments, the methods may be used to treat head, neck, breast, lung, liver, ovaries, colon, colorectal, melanoma, brain or prostate tumors. In a further embodiment, the tumor may be a human papillomavirus (HPV)-positive tumor, including but not limited to HPV+ tumors of the head or neck, and/or cervical cancer. In a further non-limiting embodiment, the HPV+ tumor of the head or neck comprises a squamous cell carcinoma. In another embodiment, the tumor is positive for a high risk HPV, such as HPV16, 18, 16, 18, 31, 33, 35, 39, 45, 51, 52, 56, 58, 59 or 68. High risk HPV subtypes all have E6 proteins that contain a C-terminal PDZ binding motif (PDZBM), which binds with PDZ domain-containing proteins, such as protein-tyrosine phosphatase non-receptor type 13 (PTPN13). The HPV16 E6 oncoprotein interacts with the cellular phosphatase and tumor suppressor, PTPN₁₃; this interaction results in degradation of PTPN₁₃. PTPN₁₃ interacts with ephrin B₁ which is also a phosphatase substrate. Ephrin B₁ is a single pass transmembrane protein ligand that binds and activates cognate Eph receptor tyrosine kinases. In addition, ephrin B₁ itself becomes phosphorylated and initiates its own downstream signaling events. In HPV-infected cells, PTPN₁₃ expression is compromised and thus ephrin B₁ phosphorylation persists and contributes to an aggressive phenotype and disease progression. Thus, in a further embodiment, the methods further comprise administering to the subject an inhibitor of the interaction between E6 and PTPN13 (E6 binds to PTPN13 at PDZBM #4 of PTPN13). Any suitable inhibitor may be used, including but not limited to peptides that compete with E6 for binding to PTPN13, or that compete with PTPN13 for binding to E6. In another embodiment, the methods may further comprise administering to the subject an inhibitor of ephrin B1 phosphorylation. Any suitable inhibitor may be used, including but not limited to Src kinase inhibitors, including but not limited to dasatanib (chemical structure shown below), or a pharmaceutically acceptable salt thereof.

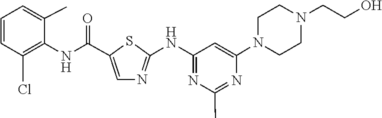 [SMILES: Cc1nc(Nc2ncc(C(=O)Nc3c(C)cccc3Cl)s2)cc(N2CCN(CCO)CC2)n1]

In another embodiment, the tumor has low PTPN13 expression levels or protein/protein activity levels, such as tumors in which PTPN13 expression levels or protein levels/activity are low due to promoter methylation, mRNA degradation, etc. In this embodiment, tumors with PTPN13 expression or protein level/activity below a threshold level (such as a control of normal levels of PTPN13 expression) are treated with the methods of the disclosure. In this embodiment, ephrin B1 phosphorylation would persist and the methods of the disclosure would be effective for treating such tumors. Exemplary tumor types with low to no PTPN13 expression include, but are not limited to, certain breast cancers (such as triple negative breast cancers: Révillion F, Puech C, Rabenoelina F, Chalbos D, Peyrat J P, Freiss G. Int J Cancer. 2009 Feb. 1; 124(3):638-43; Vermeer P D, Bell M, Lee K, Vermeer D W, Wieking B G, Bilal E, Bhanot G, Drapkin R I, Ganesan S, Klingelhutz A J, Hendriks W J, Lee J H. PLoS One. 2012; 7(1):e30447). See also Science. 2004 May 21; 304(5674):1164-6 in which PTPN13 may be mutated in colorectal, lung, breast, and gastric cancers,

The anti-human ephrin B1 antibody or fragment thereof may be administered by any suitable route, including but not limited to oral, topical, parenteral, intranasal, pulmonary, or rectal administration in dosage unit formulations containing conventional non-toxic pharmaceutically acceptable carriers, adjuvants and vehicles. In one embodiment, the anti-human ephrin B1 antibody or fragment thereof is administered via local delivery, such as by direct injection into or peri-tumorally (i.e.: adjacent to the tumor and contacting the microenvironment surrounding the tumor, both of which will have exosomes that are therapy targets).

The anti-human ephrin B1 antibody or fragment thereof may be administered in association with one or more non-toxic pharmaceutically acceptable carriers and/or diluents and/or adjuvants. The anti-human ephrin B1 antibody or fragment thereof may be administered as the sole therapy, or may be administered in combination with other therapeutic modalities (i.e.: chemotherapy, radiation therapy, surgical removal of the tumor, etc.).

### Examples

### Antibody Production

3 rats were each immunized with a DNA vector that contained the extracellular domain of human EphrinB1. Each rat received 4 genetic immunizations (once a week). On day 31, immune serum was collected and tested. Screening consisted of transfection of mammalian cells with the immunization construct containing the ECD of human EphrinB1. Serum from immunized rats was used to see if antibodies bound to the transfected mammalian cells (that had surface expression of EphrinB1's ECD). This was assessed by flow cytometry. Immune sera from all three rats was demonstrated to bind to surface expressed human EphrinB1 ECD. Rats were sacrificed and spleens isolated and fused with myeloma cells to generate the hybridomas. Fused hybridomas were cloned in 96 well dishes and screened by flow cytometry to identify clones that bind the ECD of EphrinB1 the best. 108 clones were generated; of these 54 clones had the highest binding, and 10 clones were selected for further testing. These 10 clones were expanded to T25 dishes and assessed for appropriate activity; three clones were selected for detailed analysis (BXD-1H7-G5, BXD-2G8-D3, and BXD-6C4-B9).

### Antibody Sequence Analysis

Total RNA was isolated from frozen hybridoma cell lysates in RNAlater following the technical manual of TRIzol® Reagent. Total RNA was then reverse transcribed into cDNA using isotype-specific anti-sense primers or universal primers following the technical manual of PrimeScript™ 1st Strand cDNA Synthesis Kit. The antibody fragments of VH and VL were amplified according to the standard operating procedure (SOP) of rapid amplification of cDNA ends (RACE). Amplified antibody fragments were cloned into a standard cloning vector separately. Colony PCR was performed to screen for clones with inserts of correct sizes. No less than five colonies with inserts of correct sizes were sequenced for each fragment.

### Antibody Sequences

BXD-1H7-G5
Heavy chain: Amino acids sequence (130 aa)
Leader sequence-FR1-CDR1-FR2-CDR2-FR3-CDR3-FR4
SEQ ID NO: 26)
(MAVLVLLLCLVTFPNSVLT)QIQLKETGPDLVQLTQTLSITCWSGFSLTTYNVHWVRQTPGKGLE
WMGAMWNGGRTDYNSAFKSRLSISRDTSKSQVFLNMNSLQADDTAKYFCARLDYWGQGVMVTVAS
Light chain: Amino acids sequence (127 aa)
Leader sequence-FR1-CDR1-FR2-CDR2-FR3-CDR3-FR4
(SEQ ID NO: 29)
(MESHTRVFIFLLLWLSGADG)ETVMTQSPTSMSTSIGERVTLNCKASQSVGIDVDWYQQTPGQSPK
LLIYGASNRHTGVPDRFTGSGFGRDFTLTISNVEAEDLAVYYCLHYGSIPFTFGSGTKLEIK.
BXD-2G8-D3
Heavy chain: Amino acids sequence (130 aa)
Leader sequence-FR1-CDR1-FR2-CDR2-FR3-CDR3-FR4
(SEQ ID NO: 27)
(MAVLVLLLCLVTFPNFVLT)QVQLKETGPDLVHLTQTLSITCTVSGFSLTTYNIHWVRQPPGKG
LEWMGAMWNGGGTDYNSAFKSRLSISRDTSKSQVFLKMNSLQTDDTAKYFCATPTDWGQGVMVTV
SS
Light chain: Amino acids sequence (127 aa)
Leader sequence-FR1-CDR1-FR2-CDR2-FR3-CDR3-FR4
(SEQ ID NO: 30)
(MESHTRVFIFLLLWLSGADG)ETVMTQSPTSMSTSIGERVTLNCKASQSVGINVDWYQQTPGQS
PKLLIYGASSRHTGVPDRFTGSGFGRDFTLTITNVEAEDLAWYCLHYGSIPFTFGSGTRLEIK
BXD-6C4-B9
Heavy chain: Amino acids sequence (141 aa)
Leader sequence-FR1-CDR1-FR2-CDR2-FR3-CDR3-FR4
(SEQ ID NO: 28)
(MKLWLNWIFLLTLLNGLQC)EVKLLESGGGLVQPGDSMRLSCAASGFTFTDFYMNWIRQPAGKA
PEWLGFIRNKVNGYTTDYNPSVKGRFTISRENTQNMLYLQMNTLRAEDTATYYCTREDYYYSGRF
DYWGQGVMVTVSS
Light chain: Amino acids sequence (126 aa)
Leader sequence-FR1-CDR1-FR2-CDR2-FR3-CDR3-FR4
(SEQ ID NO: 31)
(MAALQLLGLLLLWLPAMRC)DIQMTQSPSFLSASVGDRVTINCKASQNINKYLDWYHQNHGEAP
KLLIYHTNNLQTGIPSRFSGSGSGTDYTLTISSLQPEDVATYFCLQHDSRPRTFGGGTKLDLK.

### Anti-EphrinB1 Antibodies Attenuate Axonogenesis and Tumor Growth.

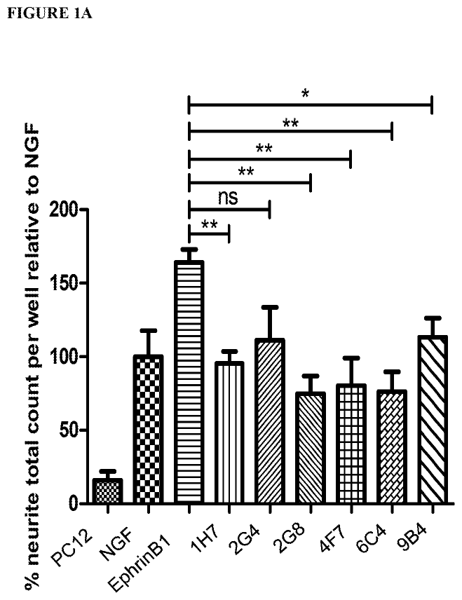

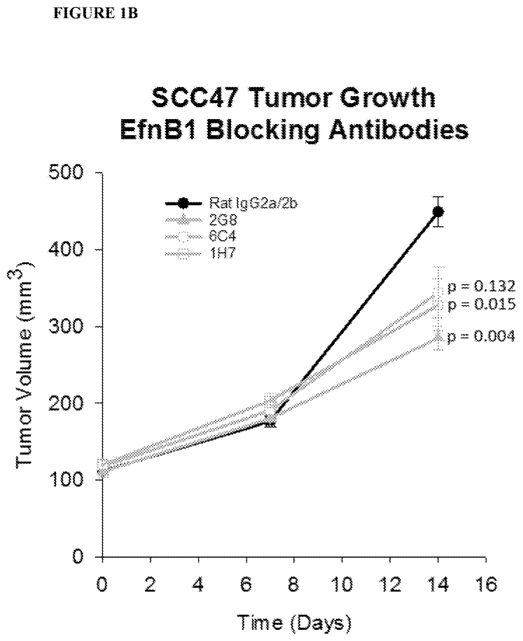

Our data support a role for exosomal EphrinB1 in potentiating axonogenesis and tumor growth. To test the utility of blocking EphrinB1 for disease control, we generated anti-human EphrinB1 antibodies as described above. We verified antibody binding to surface expressed Ephrin B1 and that the antibodies recognize human but not mouse Ephrin B1 (data not shown). To test the ability of the EphrinB1 antibodies to block neurite outgrowth in vitro, they were incubated with conditioned media from SCC47-EphrinB1 cells, which are SCC47 cells (human HPV positive head and neck squamous cell carcinoma cell line) that over-express human EphrinB1. Twenty-four hours later, the media was used to stimulate PC12 cells, which can be stimulated to differentiate into neuron-like cells, and thus provide a model to screen exosomes to induce neurite outgrowth. The next day. PC12 cells were fixed, stained for beta-III tubulin (a neuron-specific tubulin isoform) and neurite outgrowth quantified. The figure shows that all but one antibody (2G4) antibodies tested were able to significantly attenuate neurite outgrowth relative to that induced by conditioned media from SCC47-EphrinB1 (FIG. 1A). The controls in FIG. 1 are as follows: PC12=untreated PC12 cells; NGF=PC12 cells stimulated with 100 ng/ml recombinant nerve growth factor (NGF) (positive control) The number of neurites in the NGF condition is set at 100% and all other conditioned are graphed relative to that.

To test the utility of the EphrinB1 antibodies to block tumor growth, immune incompetent NOD SCID mice were implanted with human SCC47-EphrinB1 tumors. There were four groups of mice with N=5 mice/group. Two groups of mice served as controls; one control group was injected with vehicle alone while the second control group was injected with isotype matched IgG (IgG2a/2b). The other two groups were each injected with a different EphrinB1 antibody clone (1H7 or 2G8). Tumor growth was followed and mice were sacrificed at day 14 post-tumor implantation. Tumor growth of the control groups were not significantly different from each other while tumors from the EphrinB1 antibody injected groups were significantly smaller than controls (FIG. 1B). These data demonstrate that the EphrinB1 antibodies attenuate tumor growth in vivo.

### HPV Negative Disease

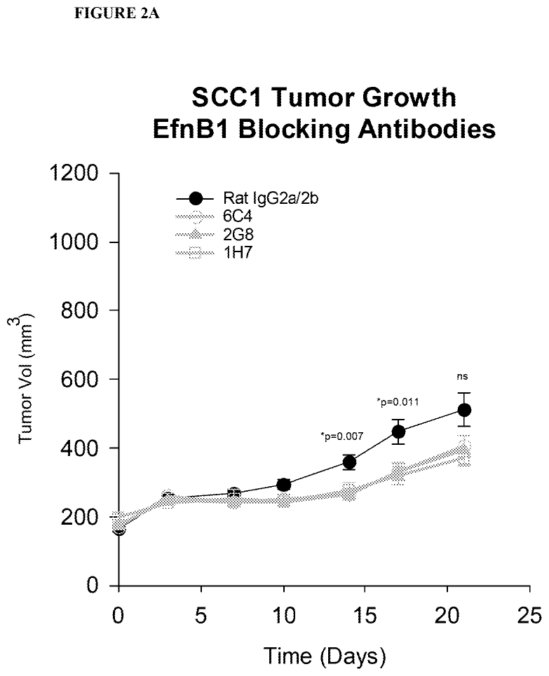

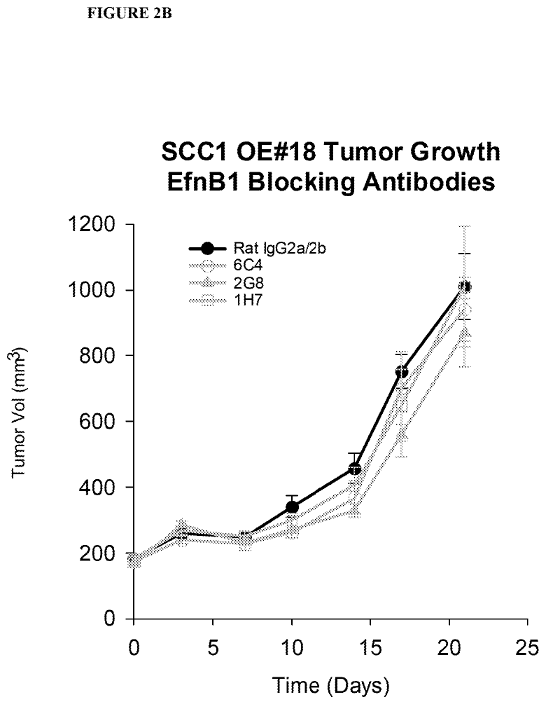

NSG mice having no immune system were injected with human SCC1 (HPV negative human head and neck squamous cell carcinoma cell line) cells that express endogenous (basal) EphrinB1 or SCC1 cells that stably over-express EphrinB1 (SCC1 OE #18). These animals were treated with 20 μg purified antibody daily by intraperitoneal injection and tumor growth followed. As shown in FIG. 2A-B below, HPV negative SCC1 cells respond to antibody treatment with decreased tumor growth, whether expressing ephrin B1 at a basal level or with ephrin B1 overexpressed.

### Epitope Mapping

For the characterization of Ephrin B1/1H7, Ephrin B1/2G8 and Ephrin B1/6C4 complexes, the measurements were performed using an Autoflex™ II MALDI ToF mass spectrometer (Bruker) equipped with CovalX's HM4 interaction module. 1 μl of the mixture obtained was mixed with 1 μl of a matrix composed of a re-crystallized sinapinic acid matrix (10 mg/ml) in acetonitrile/water (1:1, v/v), TFA 0.1% (K200 MALDI Kit). After mixing, 1 μl of each sample was spotted on the MALDI plate (SCOUT™ 384). After crystallization at room temperature, the plate was introduced in the MALDI mass spectrometer and analyzed immediately. The analysis has been repeated in triplicate

In order to determine the epitope of Ephrin B1/1H7, Ephrin B1/2G8 and Ephrin B1/6C4 complexes with high resolution, the protein complexes were incubated with deuterated cross-linkers and subjected to multi-enzymatic cleavage. After enrichment of the cross-linked peptides, the samples were analyzed by high resolution mass spectrometry (nLC-LTQ-Orbitrap™ MS) and the data generated were analyzed using XQuest™ and Stavrox™ software.

Specifically, 20 μL of Ephrin B1/1H7, Ephrin B1/2G8 or Ephrin B1/6C4 mixtures prepared were mixed with 2 μL of DSS d0/d12 (2 mg/mL; DMF) before 180 minutes incubation time at room temperature. After incubation, reaction was stopped by adding 1 μL of Ammonium Bicarbonate (20 mM final concentration) before 1 h incubation time at room temperature. Then, the solution was dried using a SPEEDVAC™ before H₂O 8M urea suspension (20 μL). After mixing, 2 μl of DTT (500 mM) were added to the solution. The mixture was then incubated 1 hour at 37° C. After incubation, 2 μl of iodoacetamide (1M) were added before 1 hour incubation time at room temperature, in a dark room. After incubation, 80 μl of the proteolytic buffer were added. The trypsin buffer contains 50 mM Ambic™ pH 8.5, 5% acetonitrile; The Chymotrypsin buffer contains Tris HCl 100 mM, CaCL2 10 mM pH 7.8; The ASP-N buffer contains Phosphate buffer 50 MM pH 7.8; The elastase buffer contains Tris HCl 50 mM pH 8.0 and the thermolysin buffer contains Tris HCl 50 mM, CaCL2 0.5 mM pH 9.0.

Epitope mapping results indicated that the human ephrin B1 epitopes for the antibodies are as follows:

1H7:
(SEQ ID NO: 33)
SWSSLNPKFLSGKG;
2G8
(SEQ ID NO: 34)
KNLEPVSWSSLNPKFLSGKG;
and
6C4
(SEQ ID NO: 35)
SGKGLVIYPKIGDKL.

Aligning these sequences against one another demonstrates that all 3 epitopes are within the human ephrin B1 sequence KNLEPVSWSSLNPKFLSGKGLVIYPKIGDKL (SEQ ID NO:32)

1. An isolated anti-human ephrin B1 antibody, or antigen-binding fragment thereof, comprising
(a) a heavy chain that comprises 1, 2, or all 3 complementarity determining regions (CDR) selected from the group consisting of:
Heavy chain CDR1 (H-CDR1) comprising the amino acid sequence selected from the group consisting of (T/D)(Y/F)(N/Y)(V/I/M)(H/N) (SEQ ID NO:1), TYN(V/I)H (SEQ ID NO:2), TYNVH (SEQ ID NO:3), TYNIH (SEQ ID NO:4), and DFYMN (SEQ ID NO:5);
Heavy chain CDR2 (H-CDR2) comprising the amino acid sequence selected from the group consisting of (F/-)(I/-)(R/-)(A/N)(M/K)(W/V)NG(G/Y)(R/G/T)TDYN(S/P)(A/S)(F/V)K(S/G) (SEQ ID NO:6), AMWNGG(R/G)TDYNSAFKS (SEQ ID NO:7), AMWNGGRTDYNSAFKS (SEQ ID NO:8), AMWNGGGTDYNSAFKS (SEQ ID NO:9), and FIRNKVNGYTTDYNPSVKG (SEQ ID NO:10); and
Heavy chain CDR3 (H-CDR3) comprising the amino acid sequence selected from the group consisting of (E/-)(D/-)(Y/-)(Y/-)(Y/-)(S/-)(G/-)(R/-)(L/P/F)(D/T)(D/Y) (SEQ ID NO:11), LDY, PTD, and EDYYYSGRFDY (SEQ ID NO:12); and
(b) a light chain that comprises 1, 2, or all 3 complementarity determining regions (CDR) selected from the group consisting of:
Light chain CDR1 (L-CDR1) comprising the amino acid sequence selected from the group consisting of KASQ(S/N)(V/I)(G/N)(I/K)(D/N/Y)(V/L)D (SEQ ID NO:13), KASQSVGI(D/N)VD (SEQ ID NO:14), KASQSVGIDVD (SEQ ID NO:15), KASQSVGINVD (SEQ ID NO:16), and KASQNINKYLD (SEQ ID NO:17);
Light chain CDR2 (L-CDR2) comprising the amino acid sequence selected from the group consisting of (G/H)(A/T)(S/N)S/N)(R/L)(H/T)T (SEQ ID NO:18), GAS(N/S)RHT (SEO ID NO:19), GASNRHT (SEQ ID NO:20), GASSRHT (SEQ ID NO:21), and HTNNLQT (SEO ID NO:22); and
Light chain CDR3 (L-CDR3): comprising the amino acid sequence selected from the group consisting of L(H/Q)(Y/H)(G/D)S(I/R)P(F/R)T (SEQ ID NO:23), LHYGSIPFT (SEQ ID NO:24), and LQHDSRPRT (SEQ ID NO:25).

2. The anti-human ephrin B1 antibody or antigen-binding fragment thereof of claim 1, wherein the heavy chain comprises 1, 2, or all 3 of the CDRS selected from the group consisting of:
H-CDR1 comprises the amino acid sequence selected from the group consisting of TYNVH (SEQ ID NO:3), TYNIH (SEQ ID NO:4), and DFYMN (SEQ ID NO:5);
H-CDR2 comprises the amino acid sequence selected from the group consisting of AMWNGGRTDYNSAFKS (SEQ ID NO:8), AMWNGGGTDYNSAFKS (SEQ ID NO:9), and FIRNKVNGYTTDYNPSVKG (SEQ ID NO:10); and
H-CDR3 comprises the amino acid sequence selected from the group consisting of LDY, PTD, and EDYYYSGRFDY (SEQ ID NO:12).

3. The anti-human ephrin B1 antibody or antigen-binding fragment thereof of claim 1, wherein the heavy chain comprises 1, 2, or all 3 of the CDRS as follows:
H-CDR1 comprises the amino acid sequence of TYNVH (SEQ ID NO:3);
H-CDR2 comprises the amino acid sequence of AMWNGGRTDYNSAFKS (SEQ ID NO:8); and
H-CDR3 comprises the amino acid sequence of LDY.

4. The anti-human ephrin B1 antibody or antigen-binding fragment thereof of claim 1, wherein the heavy chain comprises 1, 2, or all 3 of the CDRS as follows:
H-CDR1 comprises the amino acid sequence of TYNIH (SEQ ID NO:4);
H-CDR2 comprises the amino acid sequence of AMWNGGGTDYNSAFKS (SEQ ID NO:9); and
H-CDR3 comprises the amino acid sequence of PTD.

5. The anti-human ephrin B1 antibody or antigen-binding fragment thereof of claim 1, wherein the heavy chain comprises 1, 2, or all 3 of the CDRS as follows:
H-CDR1 comprises the amino acid sequence of DFYMN (SEQ ID NO:5);
H-CDR2 comprises the amino acid sequence of FIRNKVNGYTTDYNPSVKG (SEQ ID NO:10); and
H-CDR3 comprises the amino acid sequence of EDYYYSGRFDY (SEQ ID NO:12).

6. (canceled)

7. The anti-human ephrin B1 antibody, or antigen-binding fragment thereof of claim 1, wherein the light chain comprises 1, 2, or all 3 of the CDRS selected from the group consisting of:
L-CDR1 comprises the amino acid sequence selected from the group consisting of KASQSVGIDVD (SEQ ID NO:15), KASQSVGINVD (SEQ ID NO:16), and KASQNINKYLD (SEQ ID NO:17);
L-CDR2 comprises the amino acid sequence selected from the group consisting of GASNRHT (SEQ ID NO:20), GASSRHT (SEQ ID NO:21), and HTNNLQT (SEQ ID NO:22); and
L-CDR3 comprises the amino acid sequence selected from the group consisting of LHYGSIPFT(SEQ ID NO:24) and LQHDSRPRT (SEQ ID NO:25).

8. The anti-human ephrin B1 antibody, or antigen-binding fragment thereof of claim 1, wherein the light chain comprises 1, 2, or all 3 of the CDRS are as follows:
L-CDR1 comprises the amino acid sequence of KASQSVGIDVD (SEQ ID NO:15);
L-CDR2 comprises the amino acid sequence of GASNRHT (SEQ ID NO:20); and
L-CDR3 comprises the amino acid sequence of LHYGSIPFT (SEQ ID NO:24).

9. The anti-human ephrin B1 antibody, or antigen-binding fragment thereof of claim 1, wherein the light chain comprises 1, 2, or all 3 of the CDRS are as follows:
L-CDR1 comprises the amino acid sequence of KASQSVGINVD (SEQ ID NO:16);
L-CDR2 comprises the amino acid sequence of GASSRHT (SEQ ID NO:21); and
L-CDR3 comprises the amino acid sequence of LHYGSIPFT (SEQ ID NO:24).

10. The anti-human ephrin B1 antibody, or antigen-binding fragment thereof of claim 1, wherein the light chain comprises 1, 2, or all 3 of the CDRS are as follows:
L-CDR1 comprises the amino acid sequence of KASQNINKYLD (SEQ ID NO:17);
L-CDR2 comprises the amino acid sequence of HTNNLQT (SEQ ID NO:22); and
L-CDR3 comprises the amino acid sequence of LQHDSRPRT (SEQ ID NO:25).

11. (canceled)

12. The anti-human ephrin B1 antibody or antigen-binding fragment thereof of claim 1, wherein
H-CDR1 comprises the amino acid sequence selected from the group consisting of TYNVH (SEQ ID NO:3), TYNIH (SEQ ID NO:4), and DFYMN (SEQ ID NO:5);
H-CDR2 comprises the amino acid sequence selected from the group consisting of AMWNGGRTDYNSAFKS (SEQ ID NO:8), AMWNGGGTDYNSAFKS (SEQ ID NO:9), and FIRNKVNGYTTDYNPSVKG (SEQ ID NO:10);
H-CDR3 comprises the amino acid sequence selected from the group consisting of LDY, PTD, and EDYYYSGRFDY (SEQ ID NO:12);
L-CDR1 comprises the amino acid sequence selected from the group consisting of KASQSVGIDVD (SEQ ID NO:15), KASQSVGINVD (SEQ ID NO:16), and KASQNINKYLD (SEQ ID NO:17);
L-CDR2 comprises the amino acid sequence selected from the group consisting of GASNRHT (SEQ ID NO:20), GASSRHT (SEQ ID NO:21), and HTNNLQT (SEQ ID NO:22); and
L-CDR3 comprises the amino acid sequence selected from the group consisting of LHYGSIPFT(SEQ ID NO:24) and LQHDSRPRT (SEQ ID NO:25).

13. The anti-human ephrin B1 antibody or antigen-binding fragment thereof of claim 1, wherein:
H-CDR1 comprises the amino acid sequence of TYNVH (SEQ ID NO:3);
H-CDR2 comprises the amino acid sequence of AMWNGGRTDYNSAFKS (SEQ ID NO:8);
H-CDR3 comprises the amino acid sequence of LDY;
L-CDR1 comprises the amino acid sequence of KASQSVGIDVD (SEQ ID NO:15);
L-CDR2 comprises the amino acid sequence of GASNRHT (SEQ ID NO:20); and
L-CDR3 comprises the amino acid sequence of LHYGSIPFT (SEQ ID NO:24).

14. The anti-human ephrin B1 antibody or antigen-binding fragment thereof of claim 1, wherein:
H-CDR1 comprises the amino acid sequence of TYNIH (SEQ ID NO:4);
H-CDR2 comprises the amino acid sequence of AMWNGGGTDYNSAFKS (SEQ ID NO:9);
H-CDR3 comprises the amino acid sequence of PTD;
L-CDR1 comprises the amino acid sequence of KASQSVGINVD (SEQ ID NO:16);
L-CDR2 comprises the amino acid sequence of GASSRHT (SEQ ID NO:21); and
L-CDR3 comprises the amino acid sequence of LHYGSIPFT (SEQ ID NO:24).

15. The anti-human ephrin B1 antibody or antigen-binding fragment thereof of claim 1, wherein:
H-CDR1 comprises the amino acid sequence of DFYMN (SEQ ID NO:5);
H-CDR2 comprises the amino acid sequence of FIRNKVNGYTTDYNPSVKG (SEQ ID NO:10);
H-CDR3 comprises the amino acid sequence of EDYYYSGRFDY (SEQ ID NO:12);
L-CDR1 comprises the amino acid sequence of KASQNINKYLD (SEQ ID NO:17);
L-CDR2 comprises the amino acid sequence of HTNNLQT (SEQ ID NO:22); and
L-CDR3 comprises the amino acid sequence of LQHDSRPRT (SEQ ID NO:25).

16. The anti-human ephrin B1 antibody or antigen-binding fragment thereof of claim 1, comprising a heavy chain having the amino acid sequence selected from the group consisting of the following, wherein residues in parentheses are optional:
BXD-1H7-G5
Heavy chain: Amino acids sequence (130 aa)
Leader sequence-FR1-CDR1-FR2-CDR2-FR3-CDR3-FR4
(SEQ ID NO: 26)
(MAVLVLLLCLVTFPNSVLT)QIQLKETGPDLVQLTQTLSITCTVSGFSL
TTYNVHWVRQTPGKGLEWMGAMWNGGRTDYNSAFKSRLSISRDTSKSQVF
LNMNSLQADDTAKYFCARLDYWGQGVMVTVAS
BXD-2G8-D3
Heavy chain: Amino acids sequence (130 aa)
Leader sequence-FR1-CDR1-FR2-CDR2-FR3-CDR3-FR4
(SEQ ID NO: 27)
(MAVLVLLLCLVTFPNFVLT)QVQLKETGPDLVHLTQTLSITCTVSGFSL
TTYNIHWVRQPPGKGLEWMGAMWNGGGIDYNSAFKSRLSISRDTSKSQVF
LKMNSLQTDDTAKYFCATPTDWGQGVMVTVSS
BXD-6C4-B9
Heavy chain: Amino acids sequence (141 aa)
Leader sequence-FR1-CDR1-FR2-CDR2-FR3-CDR3-FR4
(SEQ ID NO: 28)
(MKLWLNWIFLLTLLNGLQC)EVKLLESGGGLVQPGDSMRLSCAASGFTF
TDFYMNWIRQPAGKAPEWLGFIRNKVNGYTTDYNPSVKGRFTISRENTQN
MLYLQMNTLRAEDTATYYCTREDYYYSGRFDYWGQGVMVTVSS

17. The anti-human ephrin B1 antibody or antigen-binding fragment thereof of claim 1, comprising a light chain having the amino acid sequence selected from the group consisting of the following, wherein residues in parentheses are optional:
BXD-1H7-G5
Light chain: Amino acids sequence (127 aa)
Leader sequence-FR1-CDR1-FR2-CDR2-FR3-CDR3-FR4
(SEQ ID NO: 29)
(MESHTRVFIFLLLWLSGADG)ETVMTQSPTSMSTSIGERVTLNCKASQS
VGIDVDWYQQTPGQSPKLLIYGASNRHTGVPDRFTGSGFGRDFTLTISNV
EAEDLAVYYCLHYGSIPFTFGSGTKLEIK
BXD-2G8-D3
Light chain: Amino acids sequence (127 aa)
Leader sequence-FR1-CDR1-FR2-CDR2-FR3-CDR3-FR4
(SEQ ID NO: 30)
(MESHTRVFIFLLLWLSGADG)ETVMTQSPTSMSTSIGERVTLNCKASQS
VGINVDWYQQTPGQSPKLLIYGASSRHTGVPDRFTGSGFGRDFTLTITNV
EAEDLAVYYCLHYGSIPFTFGSGTRLEIK
BXD-6C4-B9
Light chain: Amino acids sequence (126 aa)
Leader sequence-FR1-CDR1-FR2-CDR2-FR3-CDR3-FR4
(SEQ ID NO: 31)
(MAALQLLGLLLLWLPAMRC)DIQMTQSPSFLSASVGDRVTINCKASQNI
NKYLDWYHQNHGEAPKLLIYHTNNLQTGIPSRFSGSGSGTDYTLTISSLQ
PEDVATYFCLQHDSRPRTFGGGTKLDLK

18. The anti-human ephrin B1 antibody or antigen-binding fragment thereof of claim 1, comprising a heavy chain having the amino acid sequence below, wherein residues in parentheses are optional:
BXD-1H7-G5
Heavy chain: Amino acids sequence (130 aa)
Leader sequence-FR1-CDR1-FR2-CDR2-FR3-CDR3-FR4
(SEQ ID NO: 26)
(MAVLVLLLCLVTFPNSVLT)QIQLKETGPDLVQLTQTLSITCTVSGFSL
TTYNVHWVRQTPGKGLEWMGAMWNGGRTDYNSAFKSRLSISRDTSKSQVF
LNMNSLQADDTAKYFCARLDYWGQGVMVTVAS;
a light chain having the amino acid sequence below wherein residues in parentheses are optional:
MCD-1H7-G5
Light chain: Amino acids sequence (127 aa)
Leader sequence-FR1-CDR1-FR2-CDR2-FR3-CDR3-FR4
(SEQ ID NO: 29)
(MESHTRVFIFLLLWLSGADG)ETVMTQSPTSMSTSIGERVTLNCKASQS
VGIDVDWYQQTPGQSPKLLIYGASNRHTGVPDRFTGSGFGRDFTLTISNV
EAEDLAVYYCLHYGSIPFTFGSGTKLEIK.

19. The anti-human ephrin B1 antibody or antigen-binding fragment thereof of claim 1, comprising a heavy chain having the amino acid sequence below, wherein residues in parentheses are optional:
BXD-2G8-D3
Heavy chain: Amino acids sequence (130 aa)
Leader sequence-FR1-CDR1-FR2-CDR2-FR3-CDR3-FR4
(SEQ ID NO: 27)
(MAVLVLLLCLVTFPNFVLT)QVQLKETGPDLVHLTQTLSITCTVSGFSL
TTYNIHWVRQFPGKGLEWMGAMWNGGGIDYNSAFKSRLSISRDTSKSQVF
LKMNSLQTDDTAKYFCATPTDWGQGVMVTVSS;
and
a light chain having the amino acid sequence below, wherein residues in parentheses are optional:
BXD-2G8-D3
Light chain: Amino acids sequence (127 aa)
Leader sequence-FR1-CDR1-FR2-CDR2-FR3-CDR3-FR4
(SEQ ID NO: 30)
(MESHTRVFIFLLLWLSGADG)ETVMTQSPTSMSTSIGERVTLNCKASQS
VGINVDWYQQTPGQSPKLLIYGASSRHTGVPDRFTGSGFGRDFTLTITNV
EAEDLAVYYCLHYGSIPFTFGSGTRLEIK.

20. The anti-human ephrin B1 antibody or antigen-binding fragment thereof of claim 1, comprising a heavy chain having the amino acid sequence below, wherein residues in parentheses are optional:
BXD-6C4-B9
Heavy chain: Amino acids sequence (141 aa)
Leader sequence-FR1-CDR1-FR2-CDR2-FR3-CDR3-FR4
(SEQ ID NO: 28)
(MKLWLNWIFLLTLLNGLQC)EVKLLESGGGLVQPGDSMRLSCAASGFTF
TDFYMNWIRQPAGKAPEWLGFIRNKVNGYTTDYNPSVKGRFTISRENTQN
MLYLQMNTLRAEDTATYYCTREDYYYSGRFDYWGQGVMVTVSS;
a light chain having the amino acid sequence below, wherein residues in parentheses are optional:
BXD-6C4-B9
Light chain: Amino acids sequence (126 aa)
Leader sequence-FR1-CDR1-FR2-CDR2-FR3-CDR3-FR4
(SEQ ID NO: 31)
(MAALQLLGLLLLWLPAMRC)DIQMTQSPSFLSASVGDRVTINCKASQNI
NKYLDWYHQNHGEAPKLLIYHTNNLQTGIPSRFSGSGSGTDYTLTISSLQ
PEDVATYFCLQHDSRPRTFGGGTKLDLK.

21.-28. (canceled)

29. The anti-human ephrin B1 antibody or antigen-binding fragment thereof of claim 1, wherein the antibody comprises a monoclonal antibody, or antigen-binding fragment thereof.

30.-32. (canceled)

33. A nucleic acid encoding the anti-human ephrin B1 antibody or fragment thereof of claim 1.

34. An expression vector comprising the nucleic acid of claim 33 operatively linked to a suitable control sequence.

35. A host cell comprising the expression vector of claim 34.

36. A pharmaceutical composition, comprising
(a) the anti-human ephrin B1 antibody or fragment thereof of claim 1, or a pharmaceutically acceptable salt thereof; and
(b) a pharmaceutically acceptable carrier.

37.-39. (canceled)

40. A method for tumor treatment comprising administering to a subject having a tumor with an amount effective to limit tumor growth or metastasis of the anti-human ephrin B1 antibody or fragment thereof of claim 1.

41.-50. (canceled)
